(19) United States
(12) Patent Application Publication
(10) Pub. No.: US 2009/0028796 A1
(43) Pub. Date: Jan. 29, 2009

(54) PESTICIDAL COMPOSITION COMPRISING INDOLE DERIVATES

(75) Inventors: Pawel Bednarek, Köln (DE); Bernd Schneider, Jena (DE); Ales Svatos, Jena (DE); Paul Schulze-Lefert, Köln (DE)

Correspondence Address: CONNOLLY BOVE LODGE & HUTZ, LLP, P O BOX 2207, WILMINGTON, DE, 19899, US

(73) Assignee: Max-Planck-Gesellschaft zur Förderung der Wissenschaften e. V., München (DE)

(21) Appl. No.: 12/162,506

(22) PCT Filed: Jan. 30, 2007
(86) PCT No.: PCT/EP07/50912

§ 371 Date: Jul. 29, 2008

(30) Foreign Application Priority Data
Jan. 30, 2006 (EP) 06101052.6

(51) Int. Cl. A01N 43/40; A01P 11/00; A01P 3/00; A01P 7/04; G01N 33/53; A61K 49/00; A01N 25/00

(52) U.S. Cl. 424/9.2; 514/415; 514/418; 435/7.1; 424/405

(57) ABSTRACT

The present invention is directed to pesticidal compositions comprising indole derivatives. These indole derivatives are especially active against phytopathogenic fungi. Furthermore, the invention relates to the use of indole derivatives for the production of pesticides and the use of the compositions according to the invention as pesticides. The present invention is also directed to a process for producing a pesticidal composition and to pesticidal compositions prepared by this process. In addition, the invention relates to a process for preventing or combating pests and to a process for protecting plants against pests, especially against phytopathogenic fungi. Further, the invention is directed to plants or seeds as well as objects or materials which have been protected against pests by treatment with a composition according to the invention. Further, the invention is directed to a method for identifying a substance having pesticidal activity. Further, the invention is directed to a method of identifying the mode of action of and/or of providing binding proteins for a pesticidal compound of the present invention. Finally, the invention is directed to a method for diagnosing pest infection of a plant and to the use of a pesticidal compound of the present invention as diagnostic markers.

### FIELD OF THE INVENTION

The present invention is directed to pesticidal compositions comprising indole derivatives. These indole derivatives are especially active against phytopathogenic fungi. Furthermore, the invention relates to the use of indole derivatives for the production of pesticides and the use of the compositions according to the invention as pesticides. The present invention is also directed to a process for producing a pesticidal composition and to pesticidal compositions prepared by this process. In addition, the invention relates to a process for preventing or combating pests and to a process for protecting plants against pests, especially against phytopathogenic fungi. Further, the invention is directed to plants or seeds as well as objects or materials which have been protected against pests by treatment with a composition according to the invention. Further, the invention is directed to a method for identifying a substance having pesticidal activity. Further, the invention is directed to a method of identifying the mode of action of and/or of providing binding proteins for a pesticidal compound of the present invention. Finally, the invention is directed to a method for diagnosing pest infection of a plant and to the use of a pesticidal compound of the present invention as diagnostic markers.

### BACKGROUND OF THE INVENTION

Several pressures have accelerated the search for more environmentally and toxicologically safe and more selective and efficacious pesticides. Most commercially successful pesticides have been discovered by screening compounds synthesized in the laboratory for pesticidal properties. The average number of compounds that are screened to discover a commercially viable pesticide has increased dramatically, so that new discovery strategies are desirable. The increasing incidence of pesticide resistance is also fueling the need for new pesticides. Furthermore, most synthetic chemicals that have been commercialized as herbicides are halogenated hydrocarbons with relatively long environmental half-lives and more suspect toxicological properties than most natural compounds. Thus, natural compounds have increasingly become the focus of those interested in discovery of pesticides.

Tens of thousands of secondary products of plants have been identified, and there are estimates that hundreds of thousands of these compounds exist. These metabolites represent a large reservoir of chemical structures with biological activity. This resource is largely untapped for use as pesticides. Despite a repertoire of many antifungal and antibacterial compounds, plant products have not been used to any significant extent in the development of antimicrobial pesticides. Further knowledge of plant-derived phytoalexin elicitors could lead to their use as pesticides.

### SUMMARY OF THE INVENTION

It is an object of the present invention to provide novel pesticidal compositions.

The object is solved by the independent claims. Preferred embodiments are shown by the dependent claims.

One aspect of the present invention relates to a pesticidal composition comprising an indole derivative as pesticidally active compound. In particular, the pesticidally active compound is selected from the group consisting of 3-methylamino indole, a derivative of 3-methylamino indole, a plant metabolite which is metabolically related to 3-methylamino indole and a derivative of said plant metabolite.

In particular, the pesticidal composition according to the present invention comprises a compound of the general formula I

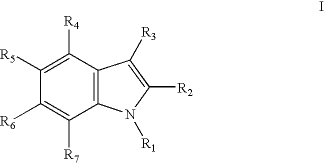

wherein
R₁ to R₇ are independently selected from the group consisting of hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylen, amine, nitro, phosphate, sulfur and oxygen; and/or
two fragments in ortho-position to each other, for example R₂ and R₃, together form another ring.

Preferably, the compounds of the general formula I are naturally-occurring plant metabolites or analogues or derivatives thereof having a relatively small environmental half-life and being less toxic than most synthetic pesticides such as halogenated hydrocarbons compounds.

Another aspect of the present invention relates to the use of a compound of the formula I

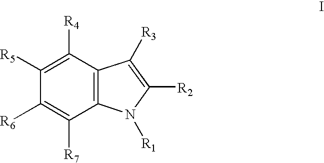

wherein R¹ to R⁷ are independently selected from the group consisting of hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylen, amine, nitro, phosphate, sulfur and oxygen; and/or two fragments in ortho-position to each other, for example R² and R³, together form another ring; as a pesticide or for the production of a pesticide.

Another aspect of the invention relates to a process for producing a pesticidal composition, wherein a compound of the formula I

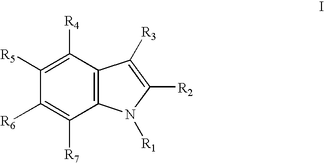

wherein R₁ to R₇ are independently selected from the group consisting of hydrogen, alkyd, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylen, amine, phosphate, nitro, sulfur and oxygen; and/or two fragments in ortho-position to each other, for example R₂ and R₃, together form another ring; is admixed with a solid or liquid carrier.

Another aspect of the present invention relates to processes for preventing or combating pests wherein the pests or their habitat or plants, seeds, soils, objects, surfaces, materials, areas or locations to be protected against the pests are treated with a pesticidal composition according to the present invention.

Another aspect of the present invention relates to a process for protecting plants against pests, especially against fingi, wherein the pests, their habitat, the plants or seeds to be protected and/or the soil in which the plants or seeds are growing are treated with a pesticidal composition according to the present invention.

Another aspect of the invention is directed to plants or seeds as well as objects or materials which have been protected against pests by treatment with a pesticidal composition according to the present invention.

Another aspect of the invention is directed to a method for identifying a substance having pesticidal activity comprising the following steps:

a) contacting a sample comprising plants or plant cells in vivo or in vitro with a compound selected from the group consisting of 3-methylamino indole, a derivative of 3-methylamino indole, a plant metabolite which is metabolically related to 3-methylamino indole and a derivative of said plant metabolite;
b) analyzing metabolites;
c) detecting whether an accumulation of a specific metabolite occurs as a consequence of the contacting of step a) by comparison with untreated samples; and
d) identifying the accumulated metabolite substance.

Further, another aspect of the invention is directed to a process for producing a pesticidal composition comprising the following steps:

a) synthesizing the substance identified in a method as described above;
b) optionally modifying the substance; and
c) admixing the optionally modified substance with a solid or liquid carrier.

Finally, another aspect of the invention is directed to a method for diagnosing pest infection of a plant, comprising the step of detecting whether an accumulation of a compound selected from the group consisting of 3-methylamino indole, a derivative of 3-methylamino indole, a plant metabolite which is metabolically related to 3-methylamino indole and a derivative of said plant metabolite and/or a compound as described above occurs in the plant by comparison with a non-infected plant.

Further exemplary embodiments of the pesticidal composition according to the present invention will be described below. However, these embodiments also apply for the use of a pesticidal compound of the present invention as a pesticide or for the production of pesticides, the process for producing a pesticidal composition, the process for preventing or combating pests, the process for protecting plants against pests, for plants or seeds as well as objects or materials which have been protected against pests by treatment with the pesticidal composition of the present invention, for a method of identifying a substance having pesticidal activity, for a method of identifying the mode of action of and/or providing binding proteins for a pesticidal compound of the present invention, for a method of diagnosing pest infection of a plant and for the use of pesticide compound of the present invention as diagnostic markers.

According to a preferred embodiment R₃ is selected from the group consisting of aminoalkylen, aldehyde, ketone, carboxyl, alkyloxycarbonyl, heterocyclus and spirocyclus.

According to a further preferred embodiment, R₁, R₂ and R₄ to R₇ are independently selected from the group consisting of hydrogen, alkyl, alkoxy, alkenyl, cycloalkyl, cycloalkenyl, aryl, heteroaryl, heterocyclic fragments, hydroxy, halogen, aldehyde, ketone, carboxy, ether, thioether, ester, thioester, phosphate, amine, nitro, sulfur and oxygen; and/or two fragments in ortho-position to each other, for example R₂ and R₃, together form another ring.

According to a preferred embodiment, R₃ is an aminomethylene having the formula II

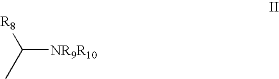

wherein R₈ to R₁₀ is selected from the group consisting of hydrogen, alkyl, alkoxy, alkenyl, cycloalkyl, cycloalkenyl, aryl, heteroaryl, heterocyclic fragments, hydroxy, halogen, aldehyde, ketone, carboxy, ether, thioether, ester, thioester, phosphate, amine, nitro, sulfur and oxygen, and are preferably hydrogen.

According to a further preferred embodiment, R₃ is an aminomethylene of the formula IIIa or IIIb

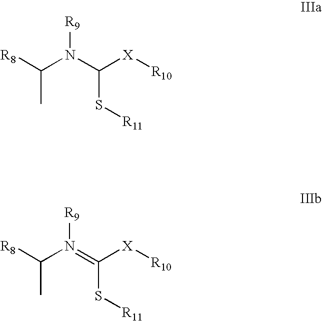

wherein
R₈ to R₁₁ is selected from the group consisting of hydrogen, alkyl, alkoxy, alkenyl, cycloalkyl, cycloalkenyl, aryl, heteroaryl, heterocyclic fragments, hydroxy, halogen, aldehyde, ketone, carboxy, ether, thioether, ester, thioester, phosphate, amine, nitro, sulfur and oxygen; and

### X is S, SO or O.

According to a further preferred embodiment, the compound has the general formula IVa or IVb

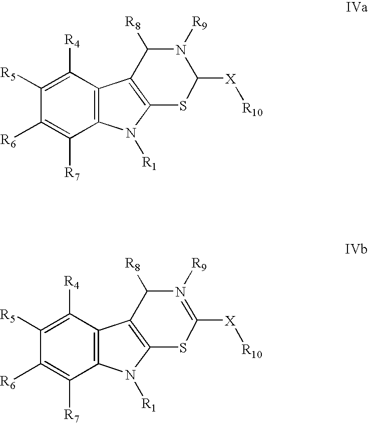

wherein
R₁ and R₄ to R₁₀ are independently selected from the group consisting hydrogen, alkyl, alkoxy, alkenyl, cycloalkyl, cycloalkenyl, aryl, heteroaryl, heterocyclic fragments, hydroxy, halogen, aldehyde, ketone, carboxy, ether, thioether, ester, thioester, phosphate, amine, nitro, sulfur and oxygen; and

### X is S or O.

According to a further preferred embodiment, R₈ and/or R₉ is hydrogen.

According to a further preferred embodiment, only one of R₁, R₂ and R₄ to R₇ is not hydrogen.

According to a further preferred embodiment, R₄ and/or R₅ are hydroxyl.

According to a further preferred embodiment, R₁, R₄ and/or R₅ are methoxy.

According to a further preferred embodiment, R₁ is methyl.

According to a further preferred embodiment, the compound is selected from one of the following compounds V to X

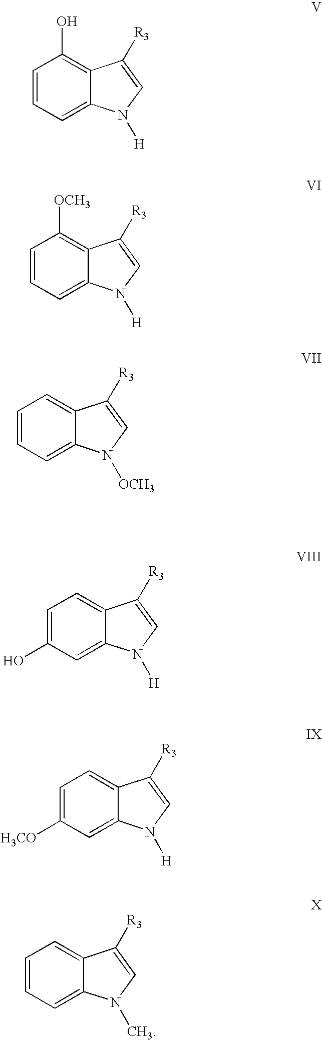

According to a further preferred embodiment, R₁, R₂ and R₄ to R₇ are hydrogen.

According to a further preferred embodiment, the compound is compound XI:

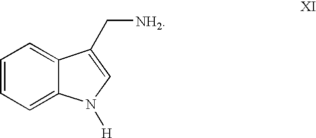

According to a further preferred embodiment, the pesticidal composition further comprises a solid or liquid carrier.

According to a further preferred embodiment, the carrier comprises an inert solid, an oil of vegetable or animal origin and/or an emulsifying or dispersing agent.

According to a further preferred embodiment, the pesticidal composition further comprises a fertilizer, growth regulator, fungicide, insecticide, bactericide, herbicide, rodenticide or other pesticide.

According to a further preferred embodiment, R₁ to R₁₁ are independently selected from the group consisting of H, OCH₃, O, OH, CH₃, S, SCH₃, SOOCH₃, NH₂, NO₂, F, Cl, Br, I, COOH, CHO, CO—COOH, COH—COOH, NHOH, CHNH₂—COOH and (CH₂)ₙR.

According to a further preferred embodiment, R₁ is H or OCH₃.

According to a further preferred embodiment, R₂ is selected from the group consisting of H, O, OH, OCH₃, S, SCH₃ and SOOCH₃.

According to a further preferred embodiment, R₃ is selected from the group consisting of H, NH₂, COOH, CHO, CO—COOH, COH—COOH, NHOH, CHNH₂—COOH and (CH₂)ₙR.

According to a further preferred embodiment, R₄ to R₇ are independently selected from the group consisting of H, OH, OCH₃, CH₃, NH₂, NO₂, F, Cl, Br and I.

Preferred substituents R according to the present invention are hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylen, amine, nitro, sulphur, phosphate and oxygen and/or derivatives thereof. Particularly preferred substituents R are H, OCH₃, O, OH, CH₃, S, SCH₃, SOOCH₃, NH₂, NO₂, F, Cl, Br, I, COOH, CHO, CO—COOH, COH—COOH, NHOH, CHNH₂—COOH and/or (CH₂)ₙR

Other objects and many of the attendant advantages of embodiments of the present invention will be readily appreciated and become better understood by reference to the following more detailed description of embodiments in connection with the accompanied drawings.

### BRIEF DESCRIPTION OF THE DRAWINGS

FIG. 1 shows constructs expressed in pen2-1 and the corresponding non-host Arabidopsis phenotype 72 hours after infection with B. graminis.

FIG. 2 shows accumulation of a compound in plant leaves 24 hours after infection with B. graminis in wild-type and pen2-1 lines:

FIG. 3 shows a mass spectrum revealing that a compound that is greatly reduced in pathogen challenged pen2 plants is 3-methylamino indole (3-MAI).

FIG. 4 shows the correlation between 3-methylamino indole accumulation and the biochemical pen2 phenotype and the non-host resistance phenotype

FIG. 5 schematically shows the tryptophan metabolism in plants including key enzymes CYP79B2 and CYP79B3.

FIG. 6 shows that CYP79B2 and CYP79B3 enzymes catalyze the key reaction in the biosynthesis of infection induces indolics such as camalexin, 3-methylamino-indole and indole-3-carboxylic acid derivatives.

FIG. 7 shows a comparison of comparison a cyp79b2 cyp79b3 double knockout line with wild type and pen2-1. It can be seen that the cyp79b2 cyp79b3 double knockout line confers enhanced disease susceptibility.

FIG. 8 shows a comparison of comparison a cyp79b2 cyp79b3 double knockout line with pad3 mutant and pen2 pad3 mutant lines. A pad3 mutant line does not accumulate camalexin. A pen2 pad3 mutant line does not accumulate camalexin and 3-methylamino-indol. A cyp79b3 double knockout line does not accumulate camalexin, 3-methylamino-indole and indole-3-carboxylic acid derivatives and confers enhanced disease susceptibility.

FIG. 9 shows the results of a leaf wash 24 hours post infection with B. graminis as described in the embodiment examples. It can be seen that 3-methylamino-indole is only secreted from leaf cells in wild type lines.

FIG. 10 show exemplary compounds of general formula I which can be used as pesticides according to the present invention.

### DETAILED DESCRIPTION OF THE INVENTION

Before describing in detail exemplary embodiments of the present invention, the following definitions are given.

The term “metabolite” refers to chemical compounds that are used in the metabolic pathways of organisms as precursors, intermediates and/or end products. Such metabolites may not only serve as chemical building units, but may also exert a regulatory activity on enzymes and their catalytic activity. It is known from the literature that such metabolites may inhibit or stimulate the activity of enzymes (Stryer, Biochemistry, (995) W.H. Freeman & Company, New York, N.Y.).

The term “metabolic pathway” is art-recognized and describes a series of reactions which take place in a wild type plant and lead to the biosynthesis of metabolites. The pathway may vary from organism to organism. The details of an organism-specific pathway can be taken from textbooks and the scientific literature. A metabolic pathway may comprise a well-known series of reactions as these are known from standard textbooks such as e.g. respiratory chain, glycosylation, tricarboxylic acid cycle, etc. Alternatively, metabolic pathways may be defined separately for the purposes of the present invention.

The term “metabolically related to 3-methylamino indole” refers to compounds which are precursors, intermediates and/or end products in the same pathway which includes the biosynthesis of metabolite 3-methylamino indole and show pesticidal activity, in particular compounds which are precursors or degradation products of 3-methylamino indole. In particular, the pathway which includes the biosynthesis of 3-methylamino indole is characterized in that the Pen 2 enzyme is involved as a key enzyme in this pathway.

The term “derivative of 3-methylamino indole” or “derivative of a plant metabolite which is metabolically related to 3-methylamino indole” refers to a compound which is derivatized or modified at any of the positions R₁ to R₁₁ as defined herein or at any other position, in particular by a substituent R as defined herein or an analogue or derivative thereof, and shows pesticidal activity.

E.g., a fungicidal activity may be determined as follows. Potentially active ingredients are dissolved in solvent in different concentrations, e.g. amounts of 0.025 and 0.01% by weight and uniformly distributed in a still liquid malt nutrient agar. The agar is poured into Petri dishes, for example having a diameter of 5 cm. After solidification of the agar, the dishes are centrally inoculated with the fungi (mycelium, spores etc.) to be analyzed, e.g. Blumeria graminis. The dishes are incubated at room temperature and the extent of the development of the fungus colony ascertained after three to five days. To this end, the number and diameter of the fungus colonies are measured and compared. Alternatively, the extent of germination can be measured.

Alternatively, blocks of wood are evenly coated with 0.2%, 0.5%, 1% and 2% (by weight) fungicide solutions and dried for several days in the air. In one series of experiments the specimens are then exposed to attack by the fungus to be analyzed, e.g. Blumeria graminis, while the specimens for a second series of experiments are placed, before exposure to the fungus, for three days in running tap water, to test the stability of the fungicide impregnation. The experiments on resistance to fungus attack are carried out in glass dishes (e.g. diameter 1.5 cm; height 3 cm), the specimens being placed upon a malt nutrient agar covered with the fungus used for test purposes and being exposed at room temperature to the fungus attack for a period of 12 weeks, fungus growth on the specimen (slight to complete cover of the specimen with fungus mycelium) is determined.

The biological action of insecticidal agents can be determined by adding the active materials in the form of solutions (e.g. in acetone) to glass vessels. By means of shaking, the walls of the glass vessels are evenly wetted by the solution, and after evaporation of the solvent, the active material remains as an even coating on the glass. Adult insects are placed in the glass vessels for 48 hours and thus exposed to the action of the active material. The mortality of the animals is determined.

Further, the pesticidal activity may be determined as follows. Leaves of plant seedlings (e.g. wheat, barley etc.) which have grown in a pot are sprayed with a suspension of the compound according to the invention in different concentrations until complete wetness of the leaves. 24 hours after drying of the spray coating, the test plants are inoculated with a spore suspension of the fungus to be analyzed. e.g. Blumeria graminis. Subsequently, the plants are cultivated in a greenhouse at a temperature of about 20-24° C. and a relative humidity of about 95-100%. After 5-7 days, the extent of the disease progression is determined visually and specified in percent infestation of the complete leaf surface. This value can then be compared to the control values of non-treated controls plants.

Within the context of the present invention a compound is considered to show pesticidal activity if pathogen entry into leaf epidermal cells and/or epiphytic hyphal growth on the leaf surface and/or conidiospore formation is inhibited by at least 50%, preferably at least 70%, more preferably at least 80% and most preferably at least 90%.

As used herein, the term “alkyl” means a linear or branched, substituted or unsubstituted saturated aliphatic hydrocarbon group having a single radical and 1-10 carbon atoms. Examples of alkyl groups include methyl, propyl, isopropyl, butyl, n-butyl, isobutyl, sec-butyl, tert-butyl and pentyl. A branched alkyl means that one or more alkyl groups such as methyl, ethyl or propyl, replace one or both hydrogens in a —CH₂ group of a linear alkyl chain. The term “lower alkyl” means an alkyl of 1-3 carbon atoms.

The term “alkoxy” means an “alkyl” as defined above connected to an oxygen radical. A particularly preferred alkoxy group is —OCH₃.

The term “aminoalkylen” includes groups such as —R′—NH₂, R′—NH—R″ or —R′—NR″R′″ wherein R′ is an alkyl, R″ and R′″ are independently selected from substituents R.

The term “aldehyd” includes groups such as —CHO or —R—CHO.

The term “ketone” includes groups such as —CO—R or —R′—CO—R″, wherein R′ and R″ are independently selected from substituents R.

The term “carboxyl” includes groups such as —COOH or —R—COOH.

The term “alkyloxycarbonyl” or “ester” includes groups such as —CO—OR, —O—COR, —R′—CO—OR″ or —R′—O—COR″, wherein R′ and R″ are independently selected from substituents R. A particularly preferred ester is —COOCH₃.

The term “thioether” includes groups such as —SR or —R′—SR″ wherein R′ and R″ are independently selected from substituents R. A particularly preferred thioether is —SCH₃.

The term “thioester” includes groups such as —SOOR and —R′—SOOR″, wherein R′ and R″ are independently selected from substituents R. A particularly preferred thioester is —SOOCH₃.

The term “cycloalkyl” means a substituted or unsubstituted non-aromatic mono- or multicyclic hydrocarbon ring system having a single radical and 3-12 carbon atoms. Exemplary monocyclic cycloalkyl rings includes cyclopropyl, cyclopentyl and cyclohexyl. Exemplary multicyclic cycloalkyl rings include adamantyl and norbornyl.

The term “aminoalkylen” means a substituted or unsubstituted alkylen connected to a substituted or unsubstituted amino group. The alkylen group includes a linear or branched saturated aliphatic hydrocarbon group having a single radical and 1-10 carbon atoms. Examples of alkylen groups include methylen, propylen, isopropylen, butylen, n-butylen, isobutylen, sec-butylen, tert-butylen and pentylen. A branched alkylen means that one or more alkyl groups such as methylen, ethylen or propylen, replace one or both hydrogens in a —CH₂— group of a linear alkylen chain. The term “lower alkylen” means an alkylen of 1-3 carbon atoms.

The term “alkenyl” means a linear or branched, substituted or unsubstituted aliphatic hydrocarbon group containing a carbon-carbon double bond having a single radical and 2-10 carbon atoms.

A “branched” alkenyl means that one or more alkyl groups such as methyl, ethyl or propyl replace one or both hydrogens in a CH₂ or —CH═ linear alkenyl chain. Exemplary alkenyl groups include ethenyl, 1- and 2-propenyl, 1-, 2- and 3-butenyl, 3-methylbut-2-enyl, 2-propenyl, heptenyl, octenyl and decenyl.

The term “cycloalkenyl” means a non-aromatic, substituted or unsubstituted monocyclic or multicyclic hydrocarbon ring system containing a carbon-carbon double bond having a single radical and 3 to 12 carbon atoms. Exemplary monocyclic cycloalkenyl rings include cyclopropenyl, cyclopentenyl, cyclohexenyl or cycloheptenyl. An exemplary multicyclic cycloalkenyl ring is norbornenyl.

The term “aryl” means a substituted or unsubstituted carbocyclic aromatic ring system containing one, two or three rings which may be attached together in a pendent manner or fused, and containing a single radical. Exemplary aryl groups include phenyl, benzyl, naphthyl and acenaphthyl.

The term “heterocyclic” or “heterocyclus” means substituted or unsubstituted cyclic compounds having one or more heteroatoms (atoms other than carbon) in the ring, and having a single radical. The ring may be saturated, partially saturated or unsaturated, and the heteroatoms may be selected from the group consisting of nitrogen, sulfur and oxygen. Examples of saturated heterocyclic radicals include D saturated 3- to 6-membered hetero-monocyclic groups containing 1 to 4 nitrogen atoms, such as pyrrolidinyl, imidazolidinyl, piperidino, piperazinyl; saturated 3- to 6-membered hetero-monocyclic groups containing 1 to 2 oxygen atoms and 1 to 3 nitrogen atoms such as morpholinyl; saturated 3- to 6-membered hetero-monocyclic groups containing 1 to 2 sulfur atoms and 1 to 3 nitrogen atoms, such as thiazolidinyl. Examples of partially saturated heterocyclic radicals include dihydrothiophene, dihydropyran and dihydrofuran. Other heterocyclic groups can be 7 to 10 carbon rings substituted with heteroatoms such as oxocanyl and thiocanyl. When the heteroatom is sulfur, the sulfur can be a sulfur dioxide such as thiocanyldioxide.

The term “heteroaryl” means substituted or unsubstituted unsaturated heterocyclic radicals, wherein “heterocyclic” is as previously described. Exemplary heteroaryl groups include unsaturated 3- to 6-membered hetero-monocyclic groups containing 1 to 4 nitrogen atoms, such as pyrrolyl, pyridyl, pyrimidyl and pyrazinyl; unsaturated condensed heterocyclic groups containing 1 to 5 nitrogen atoms, such as indolyl, quinolyl and isoquinolyl; unsaturated 3- to 6-membered hetero-monocyclic groups containing an oxygen atom, such as furyl; unsaturated 3- to 6-membered hetero-monocyclic groups containing a sulfur atom, such as thienyl; unsaturated 3- to 6-membered hetero-monocyclic groups containing 1 to 2 oxygen atoms and 1 to 3 nitrogen atoms, such as oxyzolyl; unsaturated condensed heterocyclic groups containing 1 to 2 oxygen atoms and 1 to 3 nitrogen atoms, such as benzoxazolyl; unsaturated 3- to 6-membered hetero-monocyclic group containing 1 to 2 sulfur atoms and 1 to 3 nitrogen atoms, such as thiazolyl; and unsaturated condensed heterocyclic group containing 1 to 2 sulfur atoms and 1 to 3 nitrogen atoms, such as benzothiazolyl. The term “heteroaryl” also includes unsaturated heterocyclic radicals, wherein “heterocyclic” is as previously described, in which the heterocyclic group is fused with an aryl group, in which aryl is as previously described. Exemplary fused radicals include benzofuran, benzdioxole and benzothiophene.

As used herein, the term “heterocyclic C₁₋₄ alkyl”, “heteroaromatic C₁₋₄ alkyl” and the like refer to the ring structure bonded to a C₁₋₄ alkyl radical.

The term “spirocyclus” as used herein refers to substituted or unsubstituted cyclic compounds having one or more heteroatoms (atoms other than carbon) in the ring which share one common ring member with the indole ring system. The spirocyclus may be saturated, partially saturated or unsaturated. The heteroatoms may be selected from the group consisting of nitrogen, sulfur and oxygen. Exemplary spirocyclic groups include unsaturated 3-, 4-, 5- or 6-membered heterocyclic groups containing 1 to 4 nitrogen atoms and/or 1 to 4 sulphur atoms. In particular, the spirocyclus may be substituted by a thioether group such as —SCH₃,

As used herein, the term “ring” includes cycloalkyl, cycloalkenyl, aryl, heteroaryl or heterocyclus.

All of the cyclic ring structures disclosed herein can be attached at any point where such connection is possible, as recognized by one skilled in the art.

As used herein, the term “halogen” includes fluoride, bromide, chloride, or iodide.

If one or more of R₁ to R₇ and R is O or S, this substituent is attached via a double bond.

As used herein, the term “substituted” means that one or more substituents R replace one or both hydrogens in a—CH₂— group, a ═CH— group, an —NH₂— group and/or an —NH— group.

In particular, the present invention pesticides based on plant metabolites having an indole structure which exhibit a pesticidal, especially a fungicidal effect.

Indole is an aromatic heterocyclic organic compound. It has a bicyclic structure, consisting of a six-membered benzene ring fused to a five-membered nitrogen-containing pyrrole ring. Indole can undergo electrophilic substitution, mainly at position 3, but also at the other positions (1, 2, 4, 5, 6 and 7). The indole structure is found in many organic compounds like, for example, the amino acid tryptophan. Substituted indoles are, e.g., structural elements of—and for some compounds the precursors for—tryptophan-derived alkaloids. Other indolic compounds include serotonin, indigo and the plant hormone auxin.

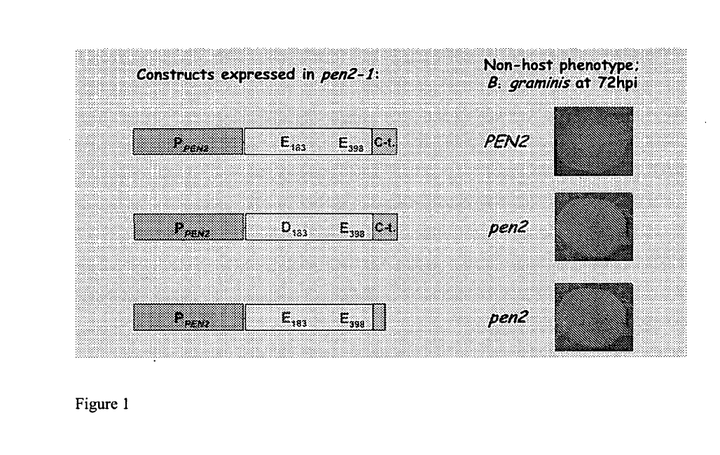

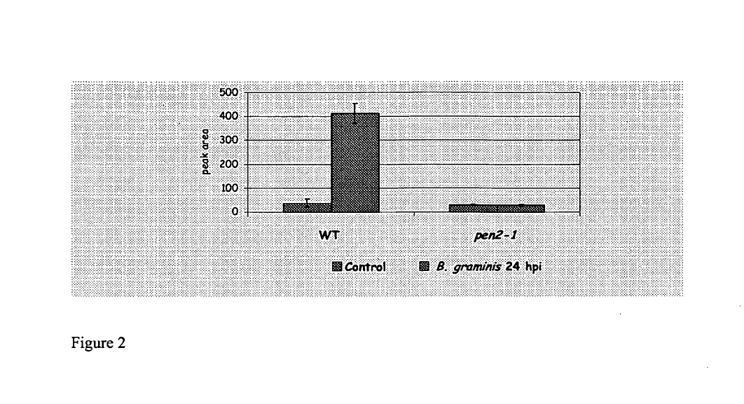

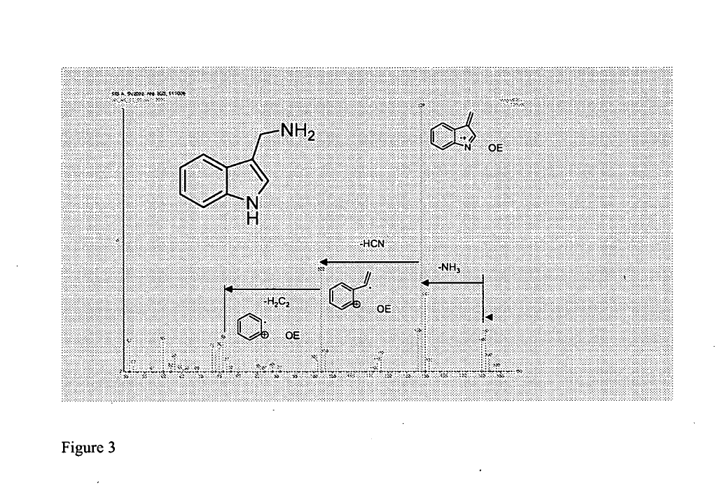

The PEN2 β-glucosidase from Arabidopsis thaliana has been recently reported (Lipka V. Science. 2005 Nov. 18; 310(5751):1180-3) as important component of plant immunity acting at the cell periphery against fungal invasion. It has been found that a compound is strongly upregulated upon pathogen treatment of wild-type plants but not in pen2 loss-of-function mutant lines (see FIGS. 1 and 2). This compound was purified by preparative HPLC, and its structure was identified as 3-methylamino indole (3-MAI) by means of NMR and mass spectroscopy (see FIG. 3). The biochemical phenotype of pen2 lines indicated that the identified metabolite must be metabolically related to the PEN2 product (aglycone).

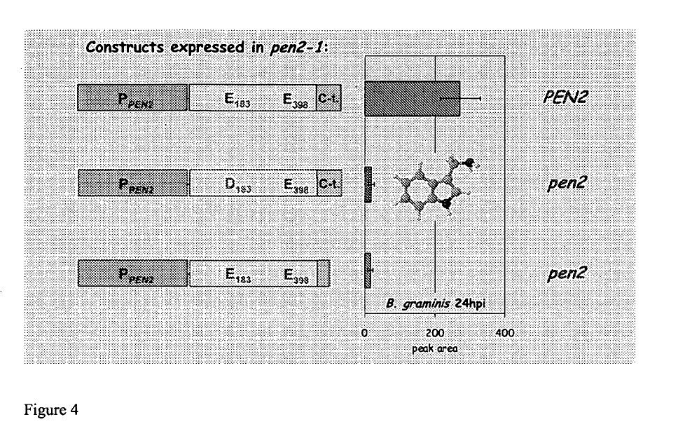

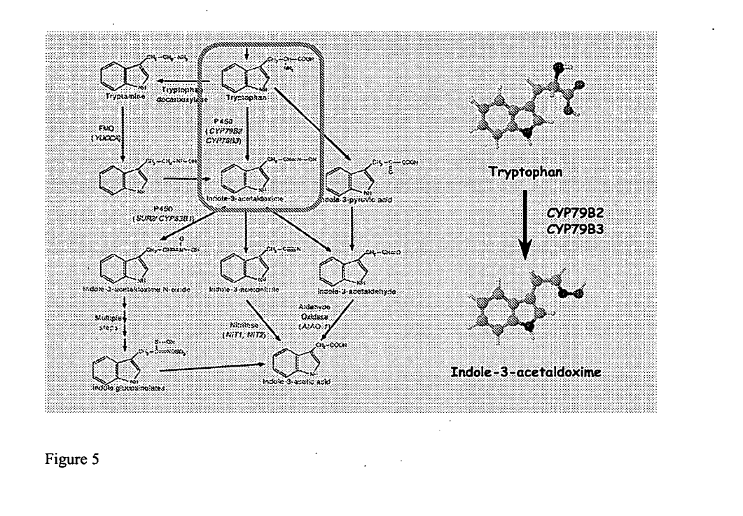

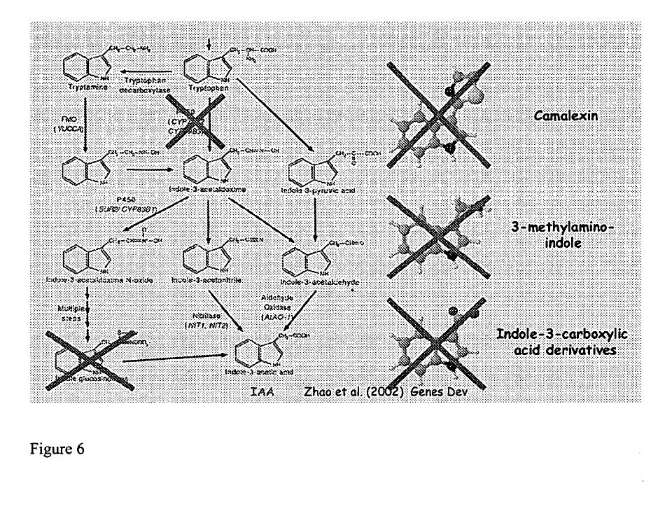

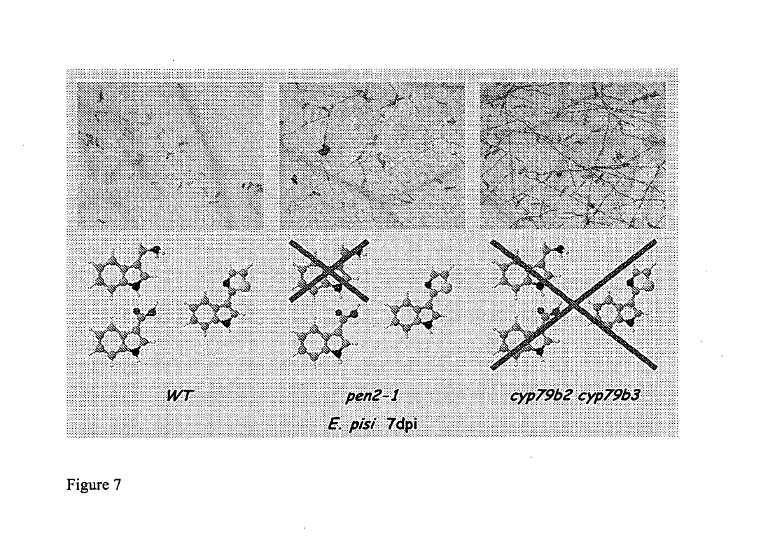

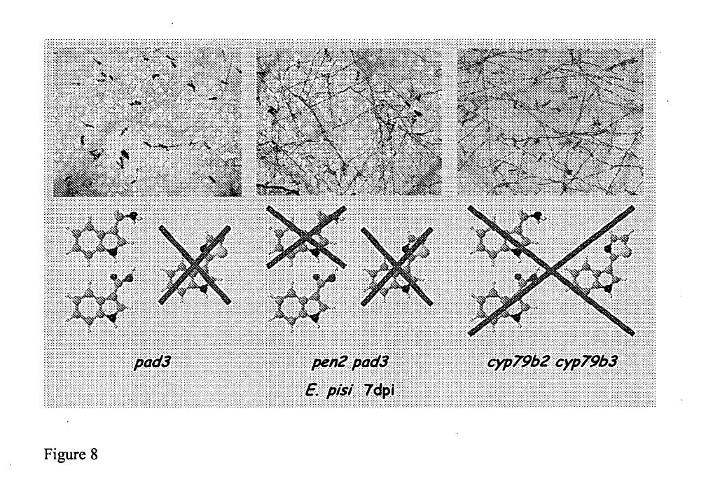

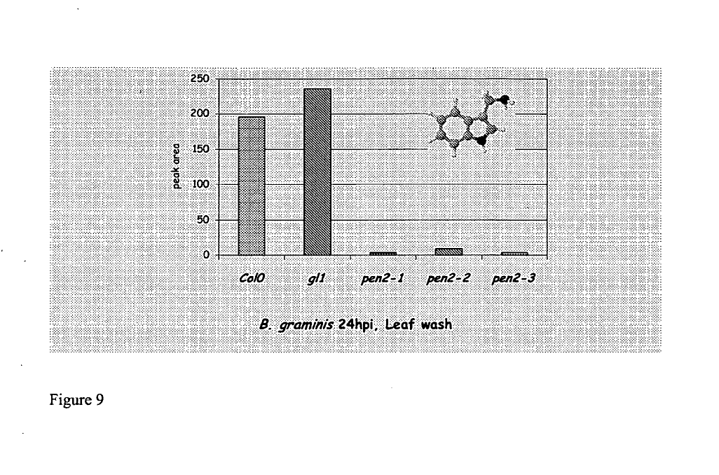

Subsequent experiments using transgenic lines expressing different variants of PEN2 (Lipka et al., vide supra) indicated a strict positive correlation between the accumulation of 3-MAI in leaf tissue and pre-invasion immunity to non-adapted fungal pathogens (non-host resistance, see FIG. 4). This correlation was further confirmed by testing cyp79b2 cyp79b3 double mutant lines that are known to affect a key step in the biosynthesis of tryptophan-derived metabolites in Arabidopsis (see FIG. 5 and Zhao Y. Genes Dev. 2002 Dec. 1; 16(23):3100-12). It has been found that cyp79b2 cyp79b3 double mutant lines fail to accumulate 3-MAI in response to pathogen challenge and are defective in pre-invasion immunity (see FIGS. 6 to 8). Moreover, 3-MAI can be detected on the leaf surface of wild-type seedlings, but not on mutant seedlings, upon pathogen challenge (see FIG. 9), indicating that this compound is secreted from plant cells.

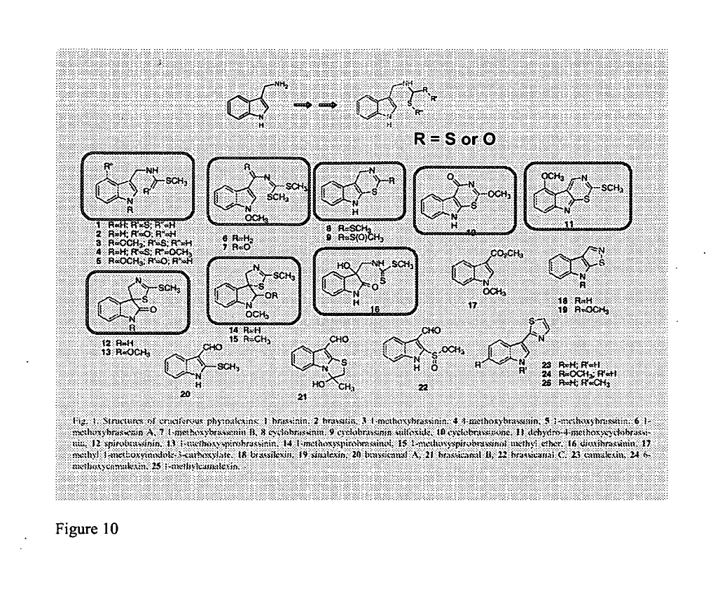

The present invention further relates to pesticidal compositions comprising indole derivatives and analogues or derivatives which are metabolically related to 3-MAI such as metabolic precursors and degradation products thereof and which show pesticidal activity. Exemplary compounds are shown in FIG. 10.

It is appreciated that substances such as indole or 3-MAI or indole derivatives which are metabolically related to 3-MAI can be modified or derivatized, and both the alternative use and the additional use of these modified or derivatized substances are embodiments of this invention. E.g., a preferred pesticidal composition according to the present invention includes 3-MAI or a derivative thereof and/or camalexin or a derivative thereof.

Preferably, pesticidal compositions according to the present invention comprise indoles being modified at position R₃. It is further preferred that modifications at residues R₁ to R₇ may be introduced separately or in combination, e.g. a combination of R₃ with one or more of R₁, R₂ and R₄ to R₇.

The modifications may comprise hydroxylated, phosphorylated and methoxylated indole derivatives and N-oxides and N-methylated indole derivatives. Furthermore, the invention relates to pesticidal compositions comprising indoles with other substituents, compounds that may be either naturally occurring or synthetic. The invention is also directed to the use of halogenated indole alkaloids.

All types of substituents, e.g., methyl, amino, nitro, fluoride, chloride, bromide, and iodide, can be introduced at the aromatic ring, e.g., at positions 4, 5, 6 and/or 7. Derivatives, conjugates and oxidation products can be formed, either as synthetic products or as the result of metabolism by living cells such as plants, microorganisms or mammalian cells. The invention further covers the use of indole derivatives which are conjugates via an ester bond, in particular with various sugars, and conjugates with amino acids and peptides.

The invention therefore refers, inter alia, to pesticidal compositions comprising at least one following indole derivatives. Underlined are naturally occurring substances; those substances not being underlined may also occur in nature. It should be understood that the source of the herein-described substances is not limited to vegetable extract. These substances can also be commercially obtained, chemically synthesized and/or biologically provided. Further, these substances may also be modified at any position by substituents R as described above.

Indole Derivatives with Substitutions at Position 3:

indole
3′-methylamino-indole 3′-MAI
indole-3-acetic acid IAA
indole-3-propionic acid
indole-3-butyric acid
indole-3-pyruvic acid IPA
tryptophan
indole-3-carboxaldehyde
indole-3-carboxylic acid
indole-3-lactic acid
indole-3-methanol
indole-3-ethanol
indole-3-acrylic acid
brassinin
indole-3-acetonitrile IAN
indole-3-glycerol-phosphate IGP
tryptamine TAM

### N-hydroxyl-tryptamine NHT

indole-3-acetaldehyde IAAld
indole-3-acetaldoxime IAOx
indole-3-acetamide IAM
indole-glucosinolate IG
1-aci-nitro-2-indolyl-ethane NIE
indole-3-methyl isothiocyanate
3′-diindolylmethane DIM

### N-methyltryptamine

### N,N-dimethyltryptamine DMT

3-methyl-3-methylamino indole
N,N-dimethyl-3-methylamino indole
3-thiazol-2′-yl-indole
malonyltryptophan
indole-3-glyoxylic acid
indole-3-acetone
indole-3-ethyl acetate
indole-3-glyoxylamide

Indole Derivatives with Substitutions at Other Positions:

indole-2-carboxylic acid
5 indole-5-carboxylic acid
4-chloro-indoleacetic acid 4-CIAA
5-hydroxyindole-3-acetic acid
5-hydroxytryptamine
5′-hydroxy-N-methyltryptamine
5′-methoxy-N,N-dimethyltryptamine
5′-methoxytryptamine
5′-methoxy-N-methyltryptamine
2,3-dioxoindolin

Cruciferous Indole Derivatives (See Also FIG. 10):

brassinin
brassitin
1-methoxybrassinin
4-methoxybrassinin
1-methoxybrassitin
1-methoxybrassenin A
1-methoxybrassenin B
cyclobrassinin
cyclobrassenin sulfoxide
cyclobrassinone
dehydro-4-methoxy cyclobrassinin
spirobrassinin
1-methoxyspirobrassinin
1-methoxyspirobrassinol
1-methoxyspirobrassinol methyl ether
dioxibrassinin
methyl 1-methoxyindole-3-carboxylate
brassilexin
sinalexin
brassicanal A
brassicanal B
brassicanal C
camalexin
6-methoxycamalexin
1-methylcamalexin

Further Indole Derivatives:

1-acetylindoline
1-methylindole
2-ethylindole
2-(4-fluorophenyl)indole
indole-2-carboxylic acid
indole-2-carboxylic acid tert-butyl ester
indole-2-carboxylic acid ethyl ester
indole-2-carboxylic acid methyl ester
indoline-2-carboxylic acid
S-(−)-indoline-2-carboxylic acid
indoline-2-sulfonic acid
2-methylindole
2-phenylindole
2-trifluoromethylindole

### L-abrine

3-acetylindole

### N-acetyl-D-tryptophan

### N-acetyl-DL-tryptophan N-Acetyl-L-tryptophan

L-alanine 3-thioindoxyl ester
2-amino-3-(3-indoxyl)-propionic acid.
BOC-L-alanine 3-thioindoxyl ester
BOC-L-phenylalanine 3-thioindoxyl ester

### BOC-tryptamine

3-(2-bromoethyl)indole
3-cyanoindole
3-diindolyl methane

### N,N-diisopropyltryptamine

### N,N-dimethyltryptamine

### N,N-dipropyltryptamine

alpha-ethyltryptamine
indole-3-acetamide
indole-3-acetic acid
indole-3-acetic acid ethyl ester
indole-3-acetic acid hydrazide
indole-3-acetic acid methyl ester
indole-3-acetone
indole-3-acetyl-L-alanine
indole-3-acetyl-DL-aspartic acid
indole-3-acetyl-L-isoleucine
indole-3-acetyl-L-leucine
indole-3-acetyl-L-phenylalanine
indole-3-acetyl-DL-tryptophan
indole-3-acetyl-L-valine
indole-3-acrylic acid
indole-3-acryloylglycine
indole-3-butyric acid
indole-3-carbinol
indole-3-carboxaldehyde
indole-3-carboxamide
indole-3-carboxylic acid
indole-3-carboxylic acid ethyl ester
indole-3-carboxylic acid methyl ester
indole-3-(N,N-dimethyl)acetamide
indole-3-(N,N-dimethyl)glyoxylamide
indole-3-glyoxylamide
indole-3-glyoxylic acid
indole-3-glyoxylic acid methyl ester
indole-3-glyoxylyl chloride
DL-indole-3-lactic acid
indole-3-methylcarbinol
indole-3-propionamide
indole-3-propionic acid
indole-3-pyruvic acid
2-(3-indolylmethyl)-L-tryptophan
3-indoxyl-3-acetate
3-indolylacetonitrile
3-indoxyl butyrate
3-indoxyl caprylate
3-indoxyl choline phosphate
3-indoxyl phosphate
3-indoxyl sulfate
3-iodo-7-azaindole
3-mercaptoindole
3-methylindole

### DL-alpha-methyltryptamine

DL-alpha-methyltryptamine monohydrochloride
DL-alpha-methyltryptamine monomethanesulfonate

### N-omega-methyltryptamine

alpha-methyl-DL-tryptophan
alpha-methyl-DL-tryptophan methyl ester
3-(2-nitrovinyl)indole
tryptamine hydrochloride

### D-tryptophan

### DL-tryptophan

### L-tryptophan

D-tryptophan methyl ester hydrochloride

### L-tryptophanol

### DL-tryptophanol

tryptophol
4-aminoindole
4-benzyloxyindole
4-bromoindole
4-chloroindole
4-cyanoindole
4-fluoroindole
4-hydroxyindole
indole-4-carboxaldehyde
indole-4-carboxylic acid
indole-4-carboxylic acid methyl ester
4-methoxyindole 4-methylindole
4-nitroindole
pindolol
5-acetylindole
5-aminoindole
5-aminoindole monohydrochloride
5-aminoindoline dihydrochloride
5-benzyloxyindole
5-bromoindole
5-chloroindole
5-cyanoindole
5-ethoxyindole
5-ethylindole
5-fluoroindole
5-hydroxyindole
indole-5-carboxaldehyde
indole-5-carboxylic acid
indole-5-carboxylic acid ethyl ester
indole-5-carboxylic acid methyl ester
5-iodoindole
5-methoxyindole
5-methylindole
5-nitroindole
5-nitroindoline
6-aminoindole
6-aminoindoline dihydrochloride
6-benzyloxyindole
6-bromoindole
6-(tert-butyldimethylsilyloxy)-indole
6-chloroindole
6-cyanoindole
6-fluoroindole
6-hydroxyindole
indole-6-carboxaldehyde
indole-6-carboxylic acid
5 indole-6-carboxylic acid methyl ester
6-methoxyindole
6-methylindole
6-nitroindole
6-nitroindoline
6-trifluoromethylindole
7-benzyloxyindole
7-bromoindole
7-chloroindole
7-(cyanomethoxy)indole
7-ethylindole
7-fluoroindole
7-hydroxyindole
indole-7-carboxaldehyde
indole-7-carboxylic acid
indole-7-carboxylic acid methyl ester
7-methoxyindole
7-methylindole
7-nitroindole

Corresponding Di- and Tri-Substituted and Higher Substituted Indole Derivatives:

### 1,2-Substituted:

1-acetylindole-2-carboxylic acid
1-acetylindoline-2-carboxylic acid
1-n-butyl-2-methylindole
1,2-dimethylindole
1-ethyl-2-phenylindole
1-methylindole-2-carboxylic acid
1-methylindole-2-carboxylic acid ethyl ester
2-methyl-1-n-octylindole

### 1,3-Substituted:

N-acetyl-3-hydroxyindole
1-acetylindole-3-carboxaldehyde
1-acetyl-3-indolinone
1-benzylindole-3-carboxylic acid
1,3-diacetylindole
1-methylindole-3-acetamide
1-methylindole-3-acetic acid
1-methylindole-3-acetic acid ethyl ester
1-methylindole-3-carboxaldehyde
1-methylindole-3-carboxylic acid
1-methyltryptamine

### 2,3-Substituted:

2,3-dicarbomethoxyindole
2,3-dimethoxyindole
2,3-dimethylindole
3-hydroxyindole-2-carboxylic acid methyl ester
isatin
2-methylgramine
2-methylindole-3-acetic acid
2-methylindole-3-carboxaldehyde
2-methyl-DL-tryptophan

### 3,4-Substituted:

4-acetoxy-N,N-diethyltryptamine
4-acetoxy-N,N-diisopropyltryptamine
4-acetoxy-N,N-dimethyltryptamine
4-benzyloxy-N,N-diisopropyltryptamine
5-benzyloxy-N,N-dimethyltryptamine
4-benzyloxyindole-3-carboxaldehyde
4-chloroindole-3-acetic acid
N,N-diethyl-4-methoxytryptamine
N,N-diisopropyl-4-hydroxytryptamine
N,N-diisopropyl-4-hydroxytryptamine hydrochloride
N,N-diisopropyl-4-methoxytryptamine
N,N-dimethyl-4-methoxytryptamine hydrochloride
4-fluorogramine
4-fluoroindole-3-acetic acid
4-fluoroindole-3-acetone
4-fluoroindole-3-acetonitrile
4-fluoroindole-3-carboxaldehyde
4-fluorotryptamine
4-fluoro-DL-tryptophan
4-methoxyindole-3-carboxaldehyde
4-methylgramine
4-methylindole-3-carboxaldehyde
4-methyl-DL-tryptophan
psilocin
psilocybine

### Tri-Substituted:

N-acetyl-5-bromo-3-hydroxyindole
L-alanine-5-bromo-4-chloro-3-indoxyl ester, trifluoroacetate salt
5-bromo-4-chloro-3-indoxyl-3-acetate
5-bromo-6-chloro-3-indoxyl-3-acetate
5-bromo-6-chloro-3-indoxyl butyrate
5-bromo-6-chloro-3-indoxyl caprate
5-bromo-4-chloro-3-indoxyl caprylate
5-bromo-6-chloro-3-indoxyl caprylate
5-bromo-4-chloro-3-indoxyl choline phosphate
5-bromo-4-chloro-3-indoxyl nonanoate
5-bromo-6-chloro-3-indoxyl nonanoate
5-bromo-4-chloro-3-indoxyl oleate
5-bromo-4-chloro-3-indoxyl palmitate
5-bromo-6-chloro-3-indoxyl palmitate

### Higher Substituted:

5-bromo-4-chloro-3-indoxyl-1-acetate
4,5,6,7-tetrafluoroindole
4,5,6,7-tetrafluoro-2-methylindole
2,3,3-trimethyl-4,5-benzo-3H-indole

Other Indole Derivatives:

4-hydroxy-IAA, 4-methoxy-IAA, 5-hydroxy-IAA, 5-methoxy-IAA, 6-hydroxy-IAA, 6-methoxy-IAA, 7-hydroxy-IAA, 7-methoxy-IAA, 6-bromoindigotin, tryptophan, 4-hydroxytryptophan, 4-methoxytryptophan, 5-hydroxytryptophan, 5-methoxytryptophan, 6-hydroxytryptophan, 6-methoxytryptophan, 7-hydroxytryptophan 7-methoxytryptophan, hypaphorine, tryptamine, 4-hydroxytryptamine, 4-methoxytryptamine, psilocin (4-hydroxy, dimethyl tryptamine), psilocybin (4-phosphate, dimethyl tryptamine), baeocystin, serotonin (5 hydroxytryptamine), 5-methoxytryptamine, bufotenine (dimethylserotonine), 0-methylbufotenine, melatonin (5-methoxy, acetamide function on tryptamineNH2), 6-5; hydroxytryptamine, 6-methoxytryptamine, 7-hydroxytryptamine, 7-methoxytryptamine, indole butyric acid, indole-3-pyruvate, indole-3-acetaldehyde, indole-3-ethanol, indole-3-aldehyde, indole-3-methanol, indole-3-carboxylic acid, 3-methylindole (skatole), indole-3-acetaldoxime, 3-methylamino indole, N-methylaminomethyl indole, indoxyls (indicans), indoleninones, 3-methylene-2-oxindole, abrine, isotan 13, isatin, indican, indigo, indurubin, indigotins 3-indolylmethyl (skatolyl), niacin, 2-oxindole-3-acetic acid, 3-methylene-2-oxindole, oxindole-3-methanol, oxindole-3-aldehyde, oxindole-3-carboxylic acid, 3-methyloxindole, acetamide, alfa-leucine, alfa-alanine, alfa-aspartate, alfa-glutamate, alfa-lysine, alfa-glycine, alfa-valine and alfa-phenylalanine, indole-3-acetonitrile, dioxindole-3-acetic acid, 3-O-beta-glucosyl-dioxindole-3-acetic acid, 7-hydroxy-2-oxindole-3-acetic acid-7′-O-beta-d-glucopyranoside, glucopyrasonyl-beta-1,4-glucopyranosyl-beta-1-N-oxindole-3-acetyl-N-aspartic acid, glucopyranosyl-beta-1-N-oxindole-3-acetyl-N-aspartic acid, 2-indolone-3-acetyl aspartic acid, 3-(O-beta-glucosyl)-2-indolone-3-acetyl aspartic acid, 3-hydroxy-2-indolone-3-acetyl aspartic acid indole-3-glycerophosphate, indole-3-glycerol, glucosinolates, such as indole-3-ylmethyl glucosinolate (glucobrassicin), 4-hydroxyindol-3-ylmethyl glucosinolate (4-hydroxyglucobrassicin), 1-acetyl-indol-3-ylmethyl glucosinolate (1-acetyl-glucobrassicin), 1-methoxyindol-3-ylmethyl glucosinolate (neoglucobrassicin), 4-methoxyindol-3-ylmethyl glucosinolate (4-methoxyglucobrassicin), 1-sulfo-indol-3-ylmethyl(glucobrassicin-1-sulfate), IAA-glucose, IAA-alfa-aspartic acid, IN-glucoside, IAA-inositol, IAA-myoinositols.

These examples are not intended to be limiting; other derivatives are commercially available at different providers, see e.g. Biosynth AG, Switzerland (www.biosynth.com), Tolly Chemicals Co., Ltd., China (www.tollychem.com or www.tollychem.com/indole.html), and Sri Krishna Pharmachem, India (www.skpcm.com or www.skpcm.com/indolecompounds.htm) which provide a detailed and extensive listing of possible indole derivatives, such as 3,5-substituted, 3,6-substituted and 3,7-substituted indoles.

The term “pesticide” or “pesticidal composition” or “pesticidal compound” as used herein means any agent, composition, substance, compound or mixture of substances intended for preventing, destroying, killing, combating, repelling, mitigating or controlling any plant or animal pest including fungi, bacteria, insects, weeds, rodents, or other organisms. The term comprises fungicides, bactericides, insecticides, herbicides, rodenticides etc. In particular, a “pesticide” or “pesticidal composition” or “pesticidal compound” as used herein means any agent, composition, substance, compound or mixture of substances which shows pesticidal activity.

A “fungicide” or “fungicidal composition” is an agent, composition, substance or mixture of substances intended for preventing, destroying, killing, combating, repelling, mitigating or controlling fungi or inhibiting their growth.

The pesticidal composition of the present invention preferably further comprises a solid or liquid carrier. According to a preferred embodiment, the carrier comprises an inert solid, an oil of vegetable or animal origin and/or an emulsifying or dispersing agent.

The substances of the present invention can be converted into the customary formulations, e.g. solutions, emulsions, dispersions, suspensions, dusts, powders, pastes and granules. The application form depends on the particular intended purpose; it is intended to ensure in any case a fine and uniform distribution of the composition according to the invention.

The compositions are prepared in a known manner, e.g. by extending the active ingredient with solvents and/or carriers, if desired using emulsifiers and dispersants. Suitable solvents, auxiliaries and carriers are essentially: water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used. Carriers such as ground natural minerals (e.g. kaolins, clays, talc, chalk) and ground synthetic minerals (e.g. highly disperse silica, silicates); emulsifiers such as nonionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable agriculturally useful salts are especially the salts of those cations or the acid addition salts of those acids whose cations and anions, respectively, have no adverse effect on the pesticidal or fungicidal action of the substances and compositions according to the invention. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium iron which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting the substances according to the invention with an acid of the corresponding anion, preferably hydrochloric acid, hydrobromic acid, sulfuric acid, phosphoric acid or nitric acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading/broadcasting and dustable products can be prepared by mixing or concomitantly grinding the active substances with a solid carrier.

Granules, for example coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, less, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active substance. The active substances are employed in a purity of from 90% to 100%, preferably 95% to 100% (according to NMR spectrum).

The following are examples of formulations:

1. Products for Dilution with Water

### A) Water-Soluble Concentrates (SL)

10 parts by weight of a compound according to the invention are dissolved in water or in a water-soluble solvent. As an alternative, wetters or other auxiliaries are added. The active compound dissolves upon dilution with water.

### B) Dispersible Concentrates (DC)

20 parts by weight of a compound according to the invention are dissolved in cyclohexanone with addition of a dispersant, for example polyvinylpyrrolidone. Dilution with water gives a dispersion.

### C) Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

### D) Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

### E) Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

### F) Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

### G) Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

### 2. Products to be Applied Undiluted

### H) Dustable Powders (DP)

5 parts by weight of a compound according to the invention are ground finely and mixed intimately with 95% of finely divided kaolin. This gives a dustable product.

### I) Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

### J) ULV Solutions (UL)

10 parts by weight of a compound according to the invention are dissolved in an organic solvent, for example xylene. This gives a product to be applied undiluted.

The active substances can be used as such, in the form of their formulations or compositions or the application forms prepared therefrom, e.g. in the form of directly sprayable solutions, powders, suspensions or dispersions, emulsions, oil dispersions, pastes, dustable products, materials for spreading, preparations for broadcasting or granules, by means of spraying, atomizing, dusting, spreading, broadcasting, watering or pouring. The application forms depend entirely on the intended purposes; it is intended to ensure in each case the finest possible distribution or dispersion of the active substances according to the invention.

Aqueous application forms can be prepared from emulsion concentrates, pastes or wettable powders (spray powders, oil dispersions) by adding water. To prepare emulsions, pastes or oil dispersions, the substances, as such or dissolved in an oil or solvent, can be homogenized in water by means of a wetting agent, tackifier, dispersant or emulsifier. Alternatively, it is possible to prepare concentrates comprising the active substance, wetting agent, tackifier, dispersant or emulsifier and, if appropriate, solvent or oil, and such concentrates are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even the active compound without additives.

Various types of oils, wetting agents, adjuvants, herbicides, fungicides, bactericides or other pesticides may be added to the active substances, if appropriate just immediately prior to use (tank mix). These agents can be admixed with the compositions according to the invention in a weight ratio of, e.g., 1:10 to 10:1.

According to a preferred embodiment, the composition of the present invention can further comprise another active substance such as a fertilizer, growth regulator, fungicide, insecticide, bactericide, herbicide, rodenticide or other pesticide. Mixing the substances or the compositions comprising these substances in the application form as pesticides, especially as fungicides, with other fungicides frequently results in a broader fungicidal spectrum of action.

In the context of the present invention, a “fertilizer” is meant to be any organic or inorganic substance or substance mixture, either of natural or synthetic origin, including manure, nitrogen, phosphorus, phosphate, potassium compounds, potash etc., which is designed for use or claimed to have value in promoting plant growth. Usually, it is spread or worked into the soil to improve the quality and/or quantity of plant growth and/or to increase the plants' fertility. Fertilizers can also hold moisture, reduce soil erosion, and improve soil structure. A fertilizer may be, e.g., ammonium sulfate, ammonium phosphate and ammonium nitrate.

In the context of the present invention, a “growth regulator” is meant to be a substance used for controlling or modifying plant growth processes or regulating the enlargement, division and/or activation of plant cells without appreciable phytotoxic effect at the dosage applied.

The following list of fungicides, together with which the substances according to the invention can be used, is intended to illustrate the possible combinations, but not to impose any limitation:
acylalanines, such as benalaxyl, metalaxyl, ofurace or oxadixyl;
amine derivatives, such as aldimorph, dodine, dodemorph, fenpropimorph, fenpropidin, guazatine, iminoctadine, spiroxamine or tridemorph;
anilinopyrimidines, such as pyrimethanil, mepanipyrim or cyprodinyl;
antibiotics, such as cycloheximide, griseofulvin, kasugamycin, natamycin, polyoxin or streptomycin;
azoles, such as bitertanol, bromoconazole, cyproconazole, difenoconazole, dinitroconazole, enilconazole, epoxiconazole, fenbuconazole, fluquinconazole, flusilazole, flutriafole, hexaconazole, imazalil, ipconazole, metconazole, myclobutanil, penconazole, propiconazole, prochloraz, prothioconazole, simeconazole, tebuconazole, tetraconazole, triadimefon, triadimenol, triflumizole or triticonazole;
dicarboximides, such as iprodione, myclozolin, procymidone or vinclozolin;
dithiocarbamates, such as ferbam, nabam, maneb, mancozeb, metam, metiram, propineb, polycarbamate, thiram, ziram or zineb;
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, penthiopyrad, picobenzamide, probenazole, proquinazid, pyrifenox, pyroquilon, quinoxyfen, silthiofam, thiabendazole, thifluzamide, thiophanate-methyl, tiadinil, tricyclazole, triforine, 5-chloro-7-(4-methylpiperidin-1-yl)-6-(2,4,6-trifluorophenyl)-[1,2,4]triazolo[1,5-a]pyrimidine;
copper fungicides, such as Bordeaux mixture, copper acetate, copper oxychloride or basic copper sulfate;
nitrophenyl derivatives, such as binapacryl, dinocap, dinobuton or nitrophthal-isopropyl;
phenylpyrroles, such as fenpiclonil or fludioxonil;
sulfur;
other fungicides, such as acibenzolar-S-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, phosphorus acid, propamocarb, phthalide, tolclofos-methyl, quintozene or zoxamide;
strobilurins, such as azoxystrobin, dimoxystrobin, enestroburin, fluoxastrobin, kresoxim-methyl, metominostrobin, orysastrobin, picoxystrobin, pyraclostrobin or trifloxystrobin;
sulfenic acid derivatives, such as captafol, captan, dichlofluanid, folpet or tolylfluanid;
cinnamides and analogous compounds, such as dimethomorph, flumetover or flumorph.

Moreover, the invention relates to the use of a substance or composition according to the present invention as pesticide, especially as fungicide, and its use for the production of a pesticide, especially a fungicide.

The present invention is also directed to a process for producing a pesticidal composition, wherein a substance according to the invention is admixed with a solid or liquid carrier. According to a preferred embodiment, the carrier comprises an inert solid, an oil of vegetable or animal origin and/or an emulsifying or dispersing agent. Furthermore, a fertilizer, growth regulator, fungicide, insecticide, bactericide, herbicide, rodenticide or other pesticide can be admixed to the composition. For further characterization of the carrier or other additives, see above.

The invention also refers to any pesticidal, preferably fungicidal, composition prepared by a process according to the invention.

The present invention is further directed to a process for preventing or combating pests, wherein the pests or their habitat or plants, seeds, soils, objects, surfaces, materials, areas or locations to be protected against the pests are treated with a composition according to the invention. The invention also relates to a process for protecting plants against pests, wherein the pests, their habitat, the plants or seeds to be protected and/or the soil in which the plants or seeds are growing are treated with a composition according to the invention.

“Pests” are any plant or animal pest including fungi, bacteria, insects, weeds, rodents, or other organisms. The “habitat,” is the place, type of site, locality, area or environment which is occupied by an organism or a population or in which the organism or population lives, grows and reproduces. F, or example, the habitat provides a plant, animal or microorganism with adequate food, water and living space.

According to a preferred embodiment, the pests to be prevented or combated are fungi. The substances and compositions according to the invention are especially suitable as fungicides. They are distinguished through an outstanding effectiveness against a broad spectrum of phytopathogenic fungi; especially from the classes of the Ascomycetes, preferably powdery mildew, most preferably Blumeria graminis, Deuteromycetes, Oomycetes and Basidiomycetes. Some are systemically effective and they can be used in plant protection as foliar fungicides, as fungicides for seed dressing and as soil fungicides.

The compounds, substances and compositions according to the invention are especially suitable for controlling the following plant diseases:

Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Bremia lactucae on lettuce,
Erysiphe cichoracearum and Sphaerotheca fuliginea on cucurbits,
Fusarium and Verticillium species on various plants,
Mycosphaerella species on cereals, bananas and peanuts,
Peronospora species on cabbage and onion plants,
Phatzopsora pachyrhizi and P. meibomiae on soy
Phytophthora infestans on potatoes and tomatoes,
Phytophthora capsici on peppers,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Pythium aphanidermatum on lawns.
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The substances and compositions according to the invention are also suitable for controlling harmful fungi, such as Paecilomyces variotii, in the protection of materials (for example wood, paper, paint dispersions, fibers or fabrics) and in the protection of stored products.

The plants to be treated with a substance or composition according to the present invention are preferably selected from the group consisting of crop plants or cultivated plants, ornamental plants and vegetables. Also, the seeds of these plants can be treated. Preferably, the plants are selected from the group consisting of crop plants such as barley, wheat, beet, cabbage, rye, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

In addition, the substances and compositions according to the present invention may also be used in plants which tolerate attack by pests, such as insects, bacteria or fungi, owing to breeding, including genetic engineering methods.

In another preferred embodiment, the treatment of the pests, especially the fungi, or their habitat or the plants, seeds, soils, objects, surfaces, materials, areas or locations to be protected against the pests is performed with a pesticidal amount of the composition.

Preferably, the substances and compositions according to the present invention are employed by treating the pests, especially the fungi, or the plants, seeds, materials or soil to be protected from pest attack with a pesticidally, especially a fungicidally effective amount of the active substance. The application can be carried out both before and after the infection of the materials, plants or seeds by the pests.

The pesticidal compositions generally comprise between 0.1 and 95%, preferably between 0.5 and 90%, by weight of active substance.

When employed in plant protection, the amounts applied are, depending on the kind of effect desired, between 0.01 and 2.0 kg of active substance per ha.

In seed treatment, amounts of active substance of 0.001 to 0.1 g, preferably 0.01 to 0.05 g, per kilogram of seed are generally necessary.

When used in the protection of objects or materials or stored products, the amount of active substance applied depends on the kind of application area and on the desired effect. Amounts customarily applied in the protection of materials are, for example, 0.001 g to 2 kg, preferably 0.005 g to 1 kg, of active substance per cubic meter of treated material.

Further, the present invention refers to plants or seeds which have been protected against pests, especially fungi, by a process according to the invention, i.e. the treatment of the plants or seeds with a composition according to the invention. The invention is also directed to any object or material, comprising wood, leather, metal, plastics, textile, paper, fibers, fabrics, paint dispersions, surface coating agents, polymer emulsions or tanning liquors, which contains or is coated with a substance or a composition according to the invention, whereby the object or material is protected against pests, especially against fungi.

Within the context of the present invention, a pesticidal, especially a fungicidal, action or activity or effect of a compound for use as a pesticide in the present invention comprises both a direct effect of the compound to pests, especially to fungi, and also an indirect effect.

Such effect may be mediated by the compounds or by one or more of its metabolites. Metabolization may be caused by metabolic pathways of the plant and/or the plant pest or pathogen. In this context the pesticidal compound of the invention can be modified by acylation, esterification, amidation, reductive alkylation, lipophilization, glycosylation, phosphorylation, arylsulphonation, alkylsulphonation, amino acid attachment and/or other ways of metabolization.

The direct or indirect pesticidal effect of the pesticidal compound of the invention and/or its metabolite may be based on the influence of the compound or its metabolite to the extent of biosynthesis or expression, e.g. in terms of transcription, translation and/or post-translational modifications, and/or activity of a protein, especially of an enzyme, which is part of the response of a plant or plant cell to pest attacks. This protein can for example be an enzyme that catalyzes a reaction of a pesticidal metabolic pathway, e.g. a reaction involved in the production or activation of phytoalexins. Alternatively or in addition, the compounds of the present invention can affect the level of a protein, especially an enzyme, by binding to DNA or RNA or to another regulatory protein, e.g. a transcription factor. One preferred way of modulating the expression of a protein-of-interest is the binding of a substance to a transcriptional regulator, nucleotide sequence capable of regulating the initiation of transcription from the promoter of the gene-of-interest, coding for a protein which is directly involved in the production or activation of phytoalexins.

The in vivo or in vitro exposure of a plant or a plant cell to a substance according to the invention then leads to the generation, activation and/or accumulation of another substance having direct pesticidal, especially fungicidal activity. The identification of this substance may be performed via mass spectrometry and/or NMR, as described herein. The pesticidal effect of this substance may be determined according to standard methods known to the person skilled in the art, such as the methods described herein.

Hence, another aspect of the present invention relates to a method for identifying a substance having pesticidal activity by metabolite profiling comprising the following steps:

a) contacting a sample comprising plants or plant cells in vivo or in vitro with a compound selected from the group consisting of 3-methylamino indole, a derivative of 3-dimethylamino indole, a plant metabolite which is metabolically related to 3-methylamino indole and a derivative of said plant metabolite;
b) analyzing metabolites;
c) detecting whether an accumulation of a specific metabolite occurs as a consequence of the contacting of step a) by comparison with untreated samples; and/or
d) identifying the accumulated metabolite substance; and

According to a preferred embodiment the compound of step a) is a compound of the general formula I

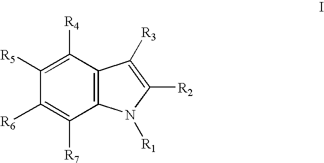

wherein
R₁ to R₇ are independently selected from the group consisting of hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylen, amine, nitro, phosphate, sulfur, and oxygen; and/or PS two fragments in ortho-position to each other, for example R₂ and R₃, together form another ring.

Optionally, the method may comprise a step e) for determining the pesticidal effect of the accumulated substance.

Within the meaning of the present invention the term “contacting” of step a) is not limited to the addition of the compound, e.g. the compound having the general formula I, to the sample but also includes increasing or decreasing the content or amount and/or biological activity of enzymes associated with the formation of the compound such as Pen2.

With respect to increasing or decreasing the content or amount and/or biological activity of an enzyme, all methods that are known in the art for increasing the amount and/or activity of a protein in a host such as the above mentioned organisms may be used. The amount of the enzyme may be increased by expression of an exogenous version of the respective protein. Further, expression of the endogenous protein can be increased by influencing the activity of the promoter and/or enhancers element and/or other regulatory activities such as phosphorylation, sumoylation, ubiquitylation etc. that regulate the activities of the respective proteins either on a transcriptional, translational or post-translational level. Besides, simply increasing the amount of e.g. the afore-mentioned enzymes, the activity of the proteins may be increased by using enzymes which carry specific mutations that allow for an increased activity of the enzyme. Such mutations may, e.g. inactivate the regions of an enzyme that are responsible for feedback inhibition. By mutating these by e.g. introducing non-conservative mutations, the enzyme does not provide for feedback regulation anymore and thus activity of the enzyme is not down-regulated if more products are produced. The mutations may be either introduced into the endogenous copy of the enzyme, or may be provided by over-expressing a corresponding mutant form of the exogenous enzyme. Such mutations may comprise point mutations, deletions or insertions. Point mutations may be conservative or non-conservative. Furthermore, deletions may comprise only two or three amino acids up to complete domains of the respective protein.

According to a further preferred embodiment the analyzing of step b) is performed by extracting soluble metabolites and performing an analytical HPLC. This is exemplified, e.g., in the embodiment examples.

Identifying in step d) of the method may be performed by NMR and/or mass spectrometry such as described herein.

As described above, the pesticidal compounds of the invention and/or their metabolites are useful to investigate their mode of action and thereby to identify new pesticidal targets. Such targets are useful to optimize the pesticidal compounds of the invention with regard to efficiency, side effects, bioavailability etc. The mode of action and/or the target of the pesticidal compound of the invention may involve both plant and/or pest genes and proteins. For example, the pesticidal compound of the invention may act through modification of gene expression in the plant and/or the pest or may modify enzymatic activities of the plant and/or the pest. Such mechanism may be assessed by methods known in the art including but not limited to:
a) gene and/or protein expression profiling: In another embodiment, the capability of the pesticidal compounds of the invention (and/or their active metabolites) to act as modulators of gene and/or expression are assessed, preferably by contacting a cell, plant, or pest with the candidate compound and analyzing the change in gene and/or protein expression. The level of gene and/or protein expression in the presence of the candidate compound is compared to the level of expression in the absence of the candidate compound. The modulated gene and/or protein can then be identified as a modulator of the pesticidal action based upon this comparison. For example, when expression of the gene and/or protein is greater (i.e., statistically significantly greater) in the presence of the candidate compound than in its absence, its overexpression might be a suitable approach to achieve pest resistance in the plant. Alternatively, when expression of the gene and/or protein is less (statistically significantly less) in the presence of the candidate compound than in suppression (e.g., by gene silencing) of said gene and/or protein might by a suitable approach to achieve pathogen resistance.
b) Two- or three-hybrid systems: In yet another aspect of the invention, the pesticidal compounds of the invention (and/or their active metabolites) can be used as “bait proteins” in a two-hybrid assay or three-hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify proteins that bind to or interact with said compounds (“binding proteins”) and/or modulate their activity. Such binding proteins can be involved in the propagation of signals by the compounds and constitute suitable targets for pesticidal compounds and are thus useful in establishing screening assays.
c) Affinity chromatography: In one embodiment, the invention provides assays for screening candidate or target proteins compounds that bind to the pesticidal compounds of the invention (and/or their active metabolites). The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.
d) Affinity photolabelling: in one embodiment, the bind proteins can also be identified in a cell-based assay in which a cell which expresses said bind proteins is treated with a pesticidal compound of the invention (or its active metabolites) which preferably carries a readily detectable label (e.g., a radioisotope or enzymatic label such that binding of the test compound to the binding protein can be determined by detecting the labeled compound in a complex). For example, test compounds can be labeled with 125I, 35S, 14C, or 3H, either directly or indirectly, and the radioisotope detected by direct counting of radio-emission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product.

Various other methods are known to the person skilled in the art to elucidate the mode of action of the pesticidal compounds provided hereunder and to identify and make use of their targets. As mentioned above, use of their targets might be both in the way of screening systems (i.e. to identify and provide new low-molecular weight pesticidal compounds) and/or in genetic engineering of the plants to achieve pathogen resistance.

Hence, another embodiment of the invention relates to a method of identifying the mode of action of a pesticidal compound of the present invention and/or of providing binding proteins for a pesticidal compound of the invention said method comprising the steps of contacting a plant, plant cell and/or a plant pathogen with a pesticidal compound of the invention or its active metabolite, and isolating the proteins specifically binding to said compound. Alternatively, said method may comprise the steps of contacting a plant, plant cell and/or a plant pathogen with a pesticidal compound of the invention or its active metabolite, and assessing the genes and/or proteins modulated in expression in consequence of said contacting.

Another aspect of the invention is directed to a process for producing a pesticidal composition comprising the following steps:

a) synthesizing the substance identified in a method according to the present invention, e.g. as described above;
b) optionally modifying the substance; and
c) admixing the optionally modified substance with a solid or liquid carrier.

Modifying in step b) may be performed as described above, e.g. with substituents R.

Finally, the pesticidal compounds of the present invention may also be used for diagnostic purposes. Pesticidal compounds as described herein can be strongly up-regulated or accumulated following pathogen challenge in plants. Hence, the pesticidal compounds of the present invention and preferably those compounds which are involved in pre-invasion immunity are especially suitable for early diagnosis of pathogen infestation. The pesticidal compounds of the present invention may, e.g., accumulate in leaf or other plant tissue and/or be secreted oil the surface of infested leaves. This accumulation may be detected and indicates the pathogen infection at an early stage. It is possible to determine the concentration or quantity of a compound according to the invention and/or to analyze the accumulation of the compound by comparison with a non-infected plant.

This diagnosis by detecting accumulation of pesticidal compounds of the present invention can be performed by means of any standard method known to the person skilled in the art. Preferably, detection methods based on the use of antibodies, preferably monoclonal antibodies, which are directed against the compound of interest may employed, such as western blot analyses, immuno-staining etc. Other suitable detection methods comprise the extraction of the soluble or surface compounds, HPLC analysis, NMR analysis and/or mass spectrometry. Exemplary detection methods are described in detail in embodiment examples 2 to 7.

If accumulation of pesticidal compounds of the present invention is detected as described above, it is possible to treat the infested plants with pesticides in order to combat the infection as early as possible, which minimizes the amount of pesticide to be used and assures a better result of pesticidal action. Further, preventive treatment of the plants with pesticides which is usually performed, e.g., when the plants have been exposed to increased humidity, can be avoided in case that no infection, i.e. no accumulation of pesticidal compounds of the present invention is detected.

Therefore, the present invention is also directed to a process for diagnosing pest infection of a plant, comprising the step of detecting whether an accumulation of a pesticidal compound according to the present invention, i.e. a compound selected from the group consisting of 3-methylamino indole, a derivative of 3-methylamino 3 indole, a plant metabolite which is metabolically related to 3-methylamino indole and a derivative of said plant metabolite, occurs in the plant by comparison with a non-infected plant.

According to a preferred embodiment, the diagnosis is performed by means of antibodies, preferably monoclonal antibodies, HPLC analysis, NMR analysis and/or mass spectrometry.

### EMBODIMENT EXAMPLES

### 1. Plant Lines and Growth Conditions

pen2-1 (see FIG. 1), pen2-2, pen2-3 knockout lines and P_{PEN2}::PEN2, P_{PEN2}::pen2_{E183D}, P_{PEN2}::pen2_{Δ28} transgenic lines were generated by Lipka et al. (vide supra); cyp79b2cyp79b3 double knockout line was kindly provided by Dr. Yunde Zhao (University of California, San Diego, USA; Zhao et al., vide supra); pad3-1 line was obtained from the Nottingham Arabidopsis Stock Center (Loughborough, UK); pad3pen2 double knockout line was a kind gift of Dr. Lore Westphal (IPB Halle, Germany). Plants were grown in growth chambers at 20-23° C. with a 12 h photoperiod.

3-4 week-old plants were inoculated with Blumeria graminis (Isolate K1) or E. pisi (Birmingham Isolate) using a settling tower. The Erysiphe pisi isolate was kindly provided by Tim Carver (Aberystwyth, UK, see Lipka et al., vide supra). For the metabolite profiling leaf rosettes were collected and use directly for sample preparation (surface metabolites) or frozen in liquid N₂ and stored at −80° C. (soluble metabolites).

### 2. Extraction of Soluble Metabolites

After addition of 50% aqueous methanol (vol/vol; 0.4 ml), the leaf tissue was homogenized using zirconia beads (1 mm; Roth, Karlsruhe, Germany) in a Mini-Beadbeater-8 (Biospec Products, Bartlesville, USA) and centrifuged for 15 min at 20,000×g. The pellets were re-extracted with 0.4 ml methanol, centrifuged again, and supernatants were combined where appropriate. The solvent was removed at 30° C. using a Speed-Vac (Eppendorf, Hamburg, Germany) and the residue was re-dissolved in 80% aqueous methanol (10 μl/4 mg initial fresh weight).

### 3. Analytical HPLC

HPLC analyses were preformed on an Agilent (Palo Alto, Calif.) 1100 HPLC system equipped with DAD and FLD detectors. Samples were analyzed on a Zorbax SB-Aq column (150/3, 3.5; Agilent) using 0.1% trifluoroacetic acid as solvent A and 98% acetonitrile/0.1% trifluoroacetic acid as solvent B at a flow rate of 0.5 ml/min at 24° C. (gradient of solvent A: 100% at 0, 94% at 3, 80% at 13, 76% at 20, 20% at 33, 0% at 34 min). In the initial experiments (metabolite profiling) respective DAD (230, 254, 273, 310, 340 nm) and FLD (ex. 275 nm, em. 350 nm; ex. 275, em. 410) chromatograms were compared in order to identify differences between WT (Col0 and gl1) and pen2 (pen2-1, pen2-2 and pen2-3) plant extracts (see FIG. 2). Once identified 3-methylamino indole was quantified based on its peak area on the FLD chromatograms (ex. 275 nm; em. 350 nm).

### 4. NMR Spectroscopy

¹H NMR, ¹H-¹H COSY, HSQC and HMBC spectra were recorded on an Avance 500 NMR spectrometer (Bruker, Karlsruhe, Germany) at 300 K using a 5 mm TXI CryoProbe™. Chemical shift values (δ) are given relative to tetramethylsilane (TMS) as an internal standard, coupling constants in Hertz (Hz). The ¹³C NMR chemical shift values were obtained from the HSQC and HMBC spectra.

### Results:

¹H NMR, ¹H-¹H COSY, HSQC and HMBC spectra were used for structure elucidation. The ¹H NMR spectrum measured in MeOH-d₄ showed signals of an AMRX spin system (δ 7.68, 7.41, 7.18, 7.16) and a singlet at δ 7.42, which together are typical of a C-3-substituted indolic compound. Another singlet (δ 4.31), integrating for two protons, was attributed to a methylene group attached to C-3 due to its HMBC correlations with C-2 (δ 126.7), C-3 (δ 107.9) and C-9 (δ 127.5). Further important HMBC correlations of protons H-2 (δ 7.42), H-4 (δ 7.68) and H-6 (δ 7.18) with the low-field angular C-8 (δ 138.4), and H-5 (δ 7.12) and H-7 (δ 7.41) with the other angular C-9 (δ 127.5) confirmed the indolic structure of the molecule. ¹H NMR and HMBC measurements in DMSO-d₆ were performed to assign exchangeable protons at the nitrogen in position 1 and aminomethylene group attached to C-3. The ¹H singlet at δ 11.20 showed HMBC cross signals with all carbons (C-2, C-3, C-9, C-8) of the pyrrol moiety, indicating direct attachment of this proton to N-1. Based on the integral of 2H, the signal at δ 7.99 was attributed to a primary amino group although the signal was too broad to show HMBC correlations with adjacent carbons. Based on these data, the structure of compound was assigned as 3-methylamino indole.

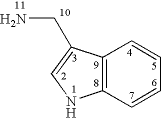

### 3-Methylamino Indole.

¹H NMR (MeOH-d₄): 7.68 (1H, ddd, J=8.0, 1.2, 0.8 Hz, 4-H); 7.42 (1H, br s, 2-H); 7.41 (1H, ddd, J=8.0, 1.0, 0.8 Hz, 7-H); 7.18 (1H, ddd, J=8.0, 7.0, 1.2 Hz, 6-H); 7.12 (1H, ddd, J=8.0, 7.0, 1.0 Hz, 5-H); 4.31 (2H, s, 10-H).

¹H NMR (DMSO-d₆): 11.2 (1H, br s, ═NH); 7.99 (2H, br s, —NH₂); 7.70 (1H, d, J=8.0 Hz, 4-H); 7.45 (1H, d, J=2.5 Hz, 2-H); 7.40 (1H, d, J=8.0 Hz, 7-H); 7.13 (1H, dd, J=8.0, 7.0 Hz, 6-H); 7.06 (1H, dd, J=8.0, 7.0 Hz, 5-H); 4.18 (2H, s, 10-H).

¹³C NMR (MeOH-d₄): δ 138.4, 8-C; 127.5, 9-C; 126.7, 2-C; 123.4, 6-C; 121.0, 5-C; 118.9, 4-C; 112.9, 7-C; 107.9, 3-C; 35.8, 10-C.

¹³C NMR (DMSO-d₆): δ 135.8, 8-C; 126.1, 9-C; 125.9, 2-C; 121.6, 6-C; 119.0, 5-C; 118.5, 4-C; 111.6, 7-C; 106.7, 3-C; 33.8, 10-C.

### 5. Mass Spectrometry and LC-MS

Chemical structures were determined by ESI-MS using a Hewlett-Packard (Avondale, Pa., USA) HP 1100 HPLC coupled to a Micromass Quattro II (Waters, Micromass, Manchester, UK) tandem quadrupole mass spectrometer (geometry quadrupole-hexapole-quadrupole) equipped with an electrospray (ESI) source. The capillary and cone voltages in ESI mode were 3.3 kV and 18 V, respectively. Nitrogen for nebulization was applied at 15 l⁻¹h⁻¹, and drying gas at 250 l⁻¹h⁻¹ and 250° C. Source and capillary were heated at 80° C. and 250° C., respectively. The mass spectrometer was operated in conventional scanning mode using the first quadrupole. Negative-ion and positive-ion full-scan mass spectra were recorded from m/z 90 to 450 (scanning time 1.5 s). Fixed precursor ion (MS/MS) spectra (daughter ion scan) were recorded by setting the first quadrupole to transmit the parent ion of interest and scanning the product ions obtained after collision of parent ions in the hexapole gas cell using the second quadrupole analyzer. Fixed product spectra (parent ion scan) were recorded by setting the second quadrupole to transmit the daughter ion of interest. Argon was used for collision-induced dissociations (CID) at 1.5×10⁻³ mbar and the collision energy was varied from 12 to 50 eV for fragmentation. Separation was achieved on a reverse-phase column (5 μm C18 phase, 250×2.1 mm i.d., Supelco) equipped with a precolumn (Supelco) using a gradient of 0.1% aqueous formic acid (A) and acetonitrile (B): 0-6 min, 2-4% B; 6-13 min, 4-18% B; 13-17 min. 18-28% B; 17-22 min, 28-53% B; 22-24 min, 53-93% B; 24-29 min, hold of 93% B (flow rate 0.4 ml min⁻¹, column temperature 30° C., UV detection at 228 nm).

### Results

The isolated samples were analyzed using LC/MS and LC/MS/MS method. The sample was dissolved in HPLC grade methanol (100 μl) and injected on LC-C₁₈ column. The fall mass scan trace show one broad peak at 6.7 min with a spectrum dominated by m/z 130 and a weak m/z 146, 147 peaks. The LC/MS/MS product scan of this ion provided mass spectrum with m/z 77 and 103, pointing to presence of aromatic ring and presumable presence of nitrogen atom. The product scan on m/z 146, another possible molecular-adduct ion provided completely different spectrum. The identity of the molecular ion was checked by product scan on m/z 130 and only m/z 147 was observed.

Molecular composition of the compound in the sample was deduced from an accurate mass EI/MS spectra; measured m/z 146.08415 corresponds to molecular compositions C₉H₁₀N₂ (calculated 146.08498) and the presence of phenyl ring deduced from product spectrum was thus confirmed (DBE=6). The loss of 17 Th from m/z 147 can be rationalized as a neutral loss of ammonia and the loss of 27 Th from m/z 130 as neutral loss of CH₂═NH. From NMR data deduced structure of 3-methylaminoindol was confirmed by a synthesis. The obtained full scan and product scan spectrum as well as retention time are undistinguishable from the natural compound. For mass spectrum see FIG. 3.

### 6. Samples of Leaf Surface Metabolites

### Detection of 3-MAI on the Leaf Surface of Wild-Type Seedlings Upon Pathogen Challenge (“Leaf Wash”)

The intact leaf rosettes were collected and dipped subsequently in hexane and in ethyl acetate (10 s in each solvent; 5 rosettes/sample). Both organic fractions were combined and solvent was removed on a rotary evaporator. The residues were re-dissolved in 50 μl 80% aqueous methanol. HPLC analyses were preformed as described above. 3-MAI accumulated on the surface of wild type plants (Col0 and gl1), but not of pen2 mutants (see FIG. 9).

### 7. Purification of 3-methylamino Indole

Leaf material (240 g) of 4 weak old A. thaliana Col0 was collected 24 h after inoculation with B. graminis and homogenized with an Ultra-Turrax homogenizer (IKA, Staufen, Germany) in 50% aq. MeOH (1000 ml), shaken at room temp. for 15 min and centrifuged for 15 min at 4,000×g. The residues were re-extracted in 80% MeOH (1000 ml) and centrifuged as above. Supernatants from both extractions were combined and concentrated on a rotary evaporator.

The compound found with analytical HPLC approach to be affected in pen2 lines was purified from the leaf extract in a three step semi-preparative approach.
separation was performed on two combined in row Atlantis C-18 columns (100/10, 5: Waters, Milford, Mass.) using 0.1% trifluoroacetic acid as solvent A and 98% acetonitrile/0.1% trifluoroacetic acid as solvent B at a flow rate of 6 ml/min at 24° C. (gradient of solvent A: 100% at 0, 94% at 3, 80% at 13, 79% at 14.75, 0% at 16 min).
the same conditions as above except solvent composition; water as solvent A and 98% acetonitrile as solvent B
separation was performed on Zorbax SB-Aq column (150/3, 3.5; Agilent) using 0.1% trifluoroacetic acid as solvent A and 98% acetonitrile/0.1% trifluoroacetic acid as solvent B at a flow rate of 0.5 ml/min at 24° C. (gradient of solvent A: 100% at 0, 94% at 3, 83.5% at 10.5, 0% at 111.5 min).

During each step fractions of column eluent were collected using a fraction collector (15 s per fraction). Fractions were rechecked with analytical HPLC for the presence of the compound of interest; positive fractions were combined, concentrated on a rotary evaporator and subjected to the subsequent step of purification. After the 3^{rd} step combined fractions rechecked with HPLC gave only a single peak of the compound of interest on the resulting chromatograms. This sample was subjected to structural identification.

### 8. Microscopic Analysis

Leaves were fixed and cleared in an ethanol/acetic acid (3:1; vol/vol) mixture. Pathogen invasion was scored microscopically; epiphytic fungal growth was visualized by staining of fungal structures with an ethanolic solution containing 0.6% Coomassie Blue (see FIG. 1).

1-44. (canceled)

45. A pesticidal composition comprising a compound selected from the group consisting of 3-methylamino indole, a derivative of 3-methylamino indole, a plant metabolite metabolically related to 3-methylamino indole, and a derivative of said metabolite metabolically related to 3-methylamino indole.

46. The pesticidal composition of claim 45, comprising a compound of general formula I 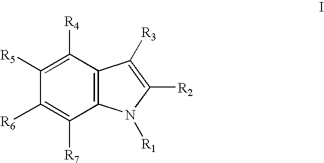
wherein
R₁, R₂, R₃, R₄, R₅, R₆, and R₇
are independently selected from the group consisting of hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylene, amine, nitro, phosphate, sulfur, and oxygen; and/or
two substituents in the ortho position to each other together define a ring system.

47. The pesticidal composition of claim 46, wherein R₃ is an aminomethylene of formula II 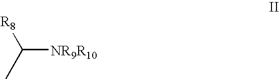
wherein
R₈, R₉, and R₁₀
are selected from the group consisting of hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylene, amine, nitro, phosphate, sulfur, and oxygen.

48. The pesticidal composition of claim 46, wherein R₃ is an aminomethylene of formulae IIIa or IIIb 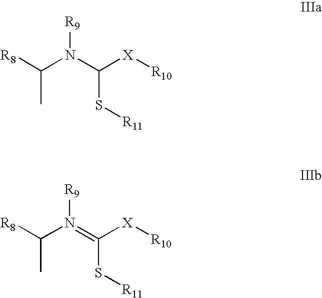
wherein
R₈, R₉, R₁₀, and R₁₁
are selected from the group consisting hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylene, amine, nitro, phosphate, sulfur, and oxygen; and
X is S, SO, or O.

49. The pesticidal composition of claim 48, wherein R₈ and/or R₉ is hydrogen.

50. The pesticidal composition of claim 45, wherein the compound has the general formulae IVa or IVb 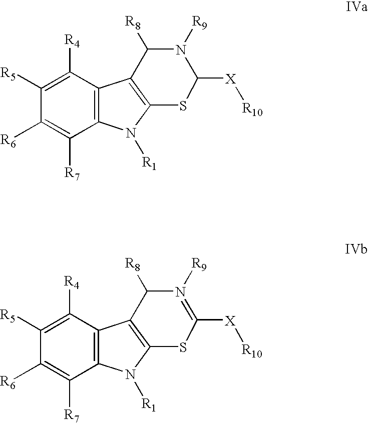
wherein
R₁, R₄, R₅, R₆, R₇, R₈, R₉, and R₁₀
are independently selected from the group consisting hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylene, amine, nitro, phosphate, sulfur, and oxygen; and
X is S or O.

51. The pesticidal composition of claim 50, wherein R₈ and/or R₉ is hydrogen.

52. The pesticidal composition of claim 45, wherein only one of R₁, R₂, R₄, R₅, R₆, and R₇ is not hydrogen.

53. The pesticidal composition of claim 45, wherein R₄ and/or R₅ are hydroxyl.

54. The pesticidal composition of claim 45, wherein R₁, R₄ and/or R₅ are methoxy.

55. The pesticidal composition of claim 45, wherein R₁ is methyl.

56. The pesticidal composition of claim 52, wherein said compound is selected from the group consisting of compounds V, VI, VII, VIII, IX, and X

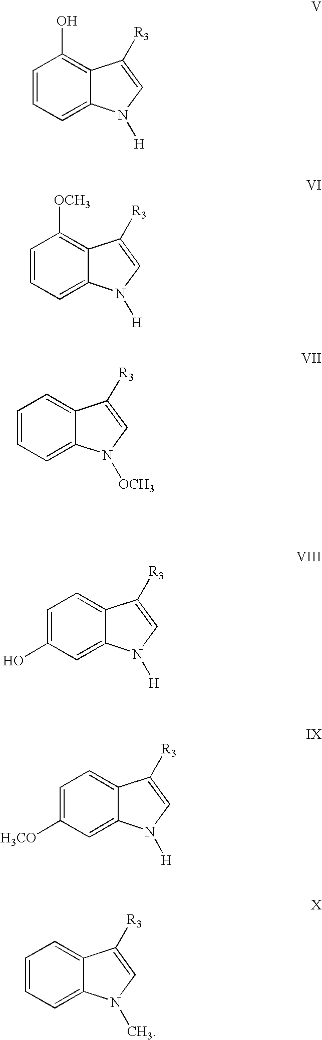

57. The pesticidal composition of claim 51, wherein R₁, R₂, R₄, R₅, R₆, and R₇ are hydrogen.

58. The pesticidal composition of claim 45, wherein said compound is compound XI

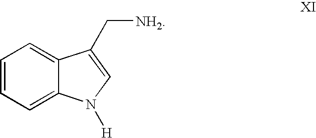

59. The pesticidal composition of claim 45, further comprising a solid or liquid carrier.

60. The pesticidal composition of claim 59, wherein said carrier comprises an inert solid, an oil of vegetable or animal origin, and/or an emulsifying or dispersing agent.

61. The pesticidal composition of claim 45, further comprising a fertilizer, a growth regulator, a fungicide, an insecticide, a bactericide, a herbicide, a rodenticide, or other pesticide.

62. A process for producing a pesticidal composition, wherein a compound selected from the group consisting of 3-methylamino indole, a derivative of 3-methylamino indole, a plant metabolite which is metabolically related to 3-methylamino indole and a derivative of said plant metabolite or a compound of general formula I 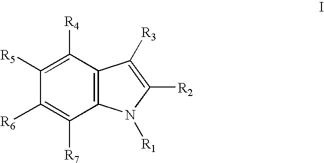
wherein
R₁, R₂, R₃, R₄, R₅, R₆, and R₇
are independently selected from the group consisting of hydrogen, alkyl, alkoxy, thioether, alkenyl, cycloalkyl, cycloalkenyl, aryl, heteroaryl, heterocyclus, spirocyclus, hydroxy, halogen, aldehyde, ketone, carboxyl, sulfonyl, ester, thioester, aminoalkylene, amine, nitro, phosphate, sulfur, and oxygen; and/or
two substituents in the ortho position to each other together define a ring system;
is admixed with a solid or liquid carrier.

63. The process of claim 62, wherein said carrier comprises an inert solid, an oil of vegetable or animal origin, and/or an emulsifying or dispersing agent.

64. The process of claim 62, further comprising admixing a fertilizer, a growth regulator, a fungicide, an insecticide, a bactericide, a herbicide, a rodenticide, or other pesticide with said composition.

65. A pesticidal composition prepared by the process of claim 62.

66. A process for preventing or combating pests, wherein said pests, the habitat of said pests, plants, seeds, soils, objects, surfaces, materials, areas, or locations to be protected against the pests are treated with the composition of claim 45.

67. A process for protecting plants against pests, wherein said pests, the habitat of said pests, the plants or seeds to be protected, and/or the soil wherein said plants or seeds are growing are treated with the composition of claim 45.

68. The process of claim 66, wherein said pests are fungi.

69. The process of claim 68, wherein said fungi are selected from the group consisting of Ascomycetes, Deuteromycetes, Oomycetes, Basidiomycetes, powdery mildew, and Blumeria graminis.

70. The process of claim 66, wherein said treatment is performed with a pesticidal amount of said composition.

71. The process of claim 66, wherein said plants or seeds are selected from the group consisting of crop plants, ornamental plants, vines, fruits, vegetables, crop plant seeds, ornamental plant seeds, vine seeds, fruit seeds, and vegetable seeds.

72. The process of claim 71, wherein said plants or seeds are selected from the group consisting of barley, wheat, beet, cabbage, rye, oats, rice, maize, grass, bananas, cotton, soya, coffee, sugar cane, vines, fruits, cucumbers, beans, tomatoes, potatoes, cucurbits, barley seeds, wheat seeds, beet seeds, cabbage seeds, rye seeds, oat seeds, rice seeds, maize seeds, grass seeds, banana seeds, cotton seeds, soya seeds, coffee seeds, sugar cane seeds, vine seeds, fruit seeds, cucumber seeds, bean seeds, tomato seeds, potato seeds, and cucurbit seeds.

73. Plants or seeds which have been protected against pests by the process of claim 66.

74. An object or material comprising wood, leather, metal, plastics, textile, paper, fibers, fabrics, paint dispersions, surface coating agents, polymer emulsions, or tanning liquors which contains or is coated with a compound selected from the group consisting of 3-methylamino indole, a derivative of 3-methylamino indole, a plant metabolite which is metabolically related to 3-methylamino indole and a derivative of said plant metabolite or with a composition according to claims 46, whereby the object or material is protected against pests.

75. A method for identifying a substance having pesticidal activity, comprising:
a) contacting a sample comprising plants or plant cells in vivo or in vitro with a compound selected from the group consisting of 3-methylamino indole, a derivative of 3-methylamino indole, a plant metabolite which is metabolically related to 3-methylamino indole, and a derivative of said plant metabolite which is metabolically related to 3-methylamino indole;
b) analyzing metabolites;
c) detecting whether accumulation of a specific metabolite occurs as a consequence of the contacting of a) by comparison with untreated samples; and
d) identifying the accumulated metabolite substance.

76. The method of claim 75, wherein the compound of a) is a compound as defined in claim 46.

77. The method of claim 75, further comprising e) determining the pesticidal effect of the accumulated substance.

78. The method of claim 75, wherein said contacting of a) is performed by over-expressing enzymes associated with the formation of the compound or by addition of the compound to the sample.

79. The method of claim 75, wherein the analyzing of b) is performed by extracting soluble metabolites and performing an analytical HPLC.

80. The method of claim 75, wherein the identifying of d) is performed by NMR and/or mass spectrometry.

81. A process for producing a pesticidal composition comprising:
a) synthesizing the substance identified in the method of claim 77;
b) optionally modifying said substance; and
c) admixing the optionally modified substance with a solid or liquid carrier.

82. A method of identifying the mode of action of a pesticidal compound selected from the group consisting of 3-methylamino indole, a derivative of 3-methylamino indole, a plant metabolite which is metabolically related to 3-methylamino indole, a derivative of said plant metabolite which is metabolically related to 3-methylamino indole, and a compound as defined in claim 46, and/or of providing binding proteins for said pesticidal compound, comprising:
a) contacting a plant, plant cell, and/or plant pathogen with the pesticidal compound or its metabolically related compound; and
b) isolating the proteins specifically binding to said compound.

83. A method of identifying the mode of action of pesticidal compounds selected from the group consisting of 3-methylamino indole, a derivative of 3-methylamino indole, a plant metabolite which is metabolically related to 3-methylamino indole, a derivative of said plant metabolite which is metabolically related to 3-methylamino indole, and a compound as defined in claim 46, and/or of providing binding proteins for said pesticidal compound, comprising:
a) contacting a plant, plant cell, and/or plant pathogen with the pesticidal compound or its metabolically related compound; and
b) assessing the genes and/or proteins modulated in expression in consequence of said contacting.

84. A method for diagnosing pest infection of a plant, comprising the step of detecting whether an accumulation of a compound selected from the group consisting of 3-methylamino indole, a derivative of 3-methylamino indole, a plant metabolite which is metabolically related to 3-methylamino indole, a derivative of said plant metabolite which is metabolically related to 3-methylamino indole, and a compound as defined in claim 46 occurs in the plant by comparison with a non-infected plant.

85. The method of claim 84, wherein the detection is performed by methods using antibodies which are directed against the compound, HPLC analysis, NMR analysis and/or mass spectrometry.

86. A diagnostic marker for diagnosing pest infection of a plant comprising a compound selected from the group consisting of 3-methylamino indole, a derivative of 3-methylamino indole, a plant metabolite which is metabolically related to 3-methylamino indole, a derivative of said plant metabolite which is metabolically related to 3-methylamino indole, and a compound as defined in claim 46.
